(19) United States
(12) Patent Application Publication
(10) Pub. No.: US 2023/0220423 A1
(43) Pub. Date: Jul. 13, 2023

(54) Extracellular Vesicles Engineered to Be Loaded with Distinct RNA Cargo for Improved Therapeutic Efficacy

(71) Applicant: Capricor, Inc., San Diego, CA (US)

(72) Inventors: Geoffrey DECOUTO, Los Angeles, CA (US); Luis RODRIGUEZ-BORLADO, Redondo Beach, CA (US); Ann-Sophie WALRAVENS, Putte (BE)

(73) Assignee: Capricor, Inc., San Diego, CA (US)

(21) Appl. No.: 18/011,041

(22) PCT Filed: Jun. 29, 2021
(86) PCT No.: PCT/US21/39716

§ 371 Date: Dec. 16, 2022

Related U.S. Application Data
(60) Provisional application No. 63/045,551, filed on Jun. 29, 2020.

(51) Int. Cl. C12N 15/88; A61K 35/28; A61K 35/34; C12N 15/11

(52) U.S. Cl. CPC C12N 15/88; A61K 35/28; A61K 35/34; C12N 15/111; C12N 2310/141; C12N 2320/32

(57) ABSTRACT

The present invention provides extrcellular vesicles, such as exosomes, engineered to be loaded with miR-345, which may be further loaded with, e.g., miR-146a and let-7b, and/or further be depleted of miR-10a and/or miR-10b. The present invention also provides an assay method, wherein the amounts of miR-345, miR146a, and let-7b in a sample of extracellular vesicles are positively associated with potency, and wherein the amount of miR-10b in a sample of extracellular vesicles is negatively associated with potency.

### CROSS REFERENCE TO RELATED APPLICATION

This application claims the benefit of priority under 35 U.S.C. § 119(e) of U.S. Provisional Patent Application Ser. No. 63/045,551, filed Jun. 29, 2020, the contents of which are incorporated herein by reference in their entireties.

### BACKGROUND OF THE INVENTION

WO/2006/052925 and US/2012/0315252 describe cardiosphere-derived cells (CDCs), their derivation from cardiospheres, and their therapeutic utility for increasing the function of a damaged or diseased heart of a mammal. WO/2005/012510 describes cardiospheres, their derivation from cardiac tissue biopsy samples, and their therapeutic utility in cell transplantation and functional repair of the myocardium. WO/2014/028493 describes is exosomes derived from CDCs and their therapeutic utility for the repair or regeneration of damaged or diseased cardiac tissue. However, it remains unknown as to what drives increased cell potency, and there is a need in the art for identifying distinct RNA cargo relevant for controlling pro-inflammatory responses, thereby paving the way for engineering cells and extracellular vesicles (EVs), such as exosomes, with a consistently high potency, such as superior regenerative capabilities.

Different regenerative capabilities in animal models have been observed between CDCs and mesenchymal stem cells (MSCs) and also between CDCs obtained from different donors, allowing to classify each donor as potent or non-potent. It has been previously described that CDCs mediate their immune-modulatory and pro-regenerative effects through the release of EVs. To identify the biomolecules in the EVs that mediate these activities, the present inventors compared EVs from potent and non-potent CDCs (CDC-EVs) and MSCs (MSC-EVs). In the present specification, the present inventors showed that the protein expression in the membrane of EVs from MSCs or from CDCs were different. A higher presence of the tetraspanin proteins CD9 and CD81 was observed in CDC-EVs as well as a lower expression of class I HLA. CDC-EVs showed a higher presence of Y-RNA molecules and miRNAs (miRs) than MSC-EVs. Potent-derived CDC-EVs showed a stronger immunomodulatory capability on macrophages when compared with EVs from non-potent CDCs and MSC-EVs both in vitro and in vivo. In addition, three miRs were identified that are differentially expressed between EVs from potent and non-potent CDCs. The present inventors discovered that, e.g., the miR-345 expression level was significantly higher in potent CDC-EVs when compared with CDC-EVs from non-potent cells, and that miR-345 was able to downregulate the expression of RelA protein, an NF-kB family member. These results open the way to engineer cells to load EVs with miRs that will improve their efficacy.

### SUMMARY OF THE INVENTION

One aspect of the present invention provides a plurality/population of engineered extracellular vesicles derived from a plurality/population of cells, wherein the extracellular vesicles comprise miR-345, and wherein the amount of miR-345 in the engineered extracellular vesicles is substantially higher than the amount of miR-345 in non-engineered extracellular vesicles derived from the cells. According to one embodiment of this aspect of the present invention, the engineered extracellular vesicles are produced by cells genetically modified to increase the amount of miR-345 in the extracellular vesicles. According to one embodiment of this aspect of the present invention, the engineered extracellular vesicles are produced by loading extracellular vesicles with miR-345. According to one embodiment of this aspect of the present invention, the extracellular vesicles are further substantially depleted of miR-10b and/or miR-10a, and the amount(s) of miR-10b and/or miR-10a in the engineered extracellular vesicles is/are substantially lower than the amount(s) of miR-10b and/or miR-10a in non-engineered extracellular vesicles derived from the cells.

Another aspect of the present invention provides a plurality/population of engineered extracellular vesicles derived from a plurality/population of cells, wherein the extracellular vesicles comprise miR-345 and miR-146a, and wherein the amounts of the miR-345 and miR-146a in the engineered extracellular vesicles are substantially higher than the amounts of miR-345 and miR-146a in non-engineered extracellular vesicles derived from the cells. According to one embodiment of this aspect of the present invention, the engineered extracellular vesicles are produced by cells genetically modified to increase the amounts of miR-345 and miR-146a in the extracellular vesicles. According to one embodiment of this aspect of the present invention, the engineered extracellular vesicles are produced by loading extracellular vesicles with miR-345 and miR-146a. According to one embodiment of this aspect of the present invention, the extracellular vesicles are further substantially depleted of miR-10b and/or miR-10a, and the amount(s) of miR-10b and/or miR-10a in the engineered extracellular vesicles is/are substantially lower than the amount(s) of miR-10b and/or miR-10a in non-engineered extracellular vesicles derived from the cells.

Another aspect of the present invention provides a plurality/population of engineered extracellular vesicles derived from a plurality/population of cells, wherein the extracellular vesicles comprise miR-345 and let-7b, and wherein the amounts of miR-345 and let-7b in the engineered extracellular vesicles are substantially higher than the amounts of miR-345 and let-7b in non-engineered extracellular vesicles derived from the cells. According to one embodiment of this aspect of the present invention, the engineered extracellular vesicles are produced by cells genetically modified to increase the amounts of miR-345 and let-7b in the extracellular vesicles. According to one embodiment of this aspect of the present invention, the engineered extracellular vesicles are produced by loading extracellular vesicles with miR-345 and let-7b. According to one embodiment of this aspect of the present invention, the extracellular vesicles are further substantially depleted of miR-10b and/or miR-10a, and the amount(s) of miR-10b and/or miR-10a in the engineered extracellular vesicles is/are is substantially lower than the amount(s) of miR-10b and/or miR-10a in non-engineered extracellular vesicles derived from the cells.

Another aspect of the present invention provides a plurality/population of engineered extracellular vesicles derived from a plurality/population of cells, wherein the extracellular vesicles comprise miR-345, miR-146a, and let7b, and wherein the amounts of miR-345, miR-146a, and let-7b in the engineered extracellular vesicles are substantially higher than the amounts of miR-345, miR-146a, and let-7b in non-engineered extracellular vesicles derived from the cells. According to one embodiment of this aspect of the present invention, the engineered extracellular vesicles are produced by cells genetically modified to increase the amounts of miR-345, miR-146a, and let-7b in the extracellular vesicles. According to one embodiment of this aspect of the present invention, the engineered extracellular vesicles are produced by loading extracellular vesicles with miR-345, miR-146a, and let-7b. According to one embodiment of this aspect of the present invention, the extracellular vesicles are further substantially depleted of miR-10b and/or miR-10a, and the amount(s) of miR-10b and/or miR-10a in the engineered extracellular vesicles is/are substantially lower than the amount(s) of miR-10b and/or miR-10a in non-engineered extracellular vesicles derived from the cells.

Another aspect of the present invention provides a method of producing engineered extracellular vesicles, comprising genetically modifying a plurality/population of cells to overexpress miR-345, whereby the engineered extracellular vesicles produced by the cells comprise a substantially greater amount of miR-345 than extracellular vesicles produced by cells not so genetically modified. Another aspect of the present invention provides a method of producing engineered extracellular vesicles, comprising loading extracellular vesicles with miR-345, whereby the engineered extracellular vesicles comprise a substantially greater amount of miR-345 than otherwise the same extracellular vesicles not so loaded. According to one embodiment of this aspect of the present invention, the amount(s) of miR-10b and/or miR-10a in the engineered extracellular vesicles is/are substantially lower than the amount(s) of miR-10b and/or miR-10a in non-engineered extracellular vesicles.

Another aspect of the present invention provides a method of producing engineered extracellular vesicles, comprising genetically modifying a plurality/population of cells to overexpress miR-345 and miR-146, whereby the engineered extracellular vesicles produced by the cells comprise substantially greater amounts of miR-345 and miR-146 than extracellular vesicles produced by cells not so genetically modified. Another aspect of the present invention provides a method of producing engineered extracellular vesicles, comprising loading a plurality/population of extracellular vesicles with miR-345 and miR-146a, whereby the engineered extracellular vesicles comprise substantially greater amounts of miR-345 and miR-146a than otherwise the same extracellular vesicles not so loaded. According to one embodiment of this aspect of the present invention, the amount(s) of miR-10b and/or miR-10a in the engineered extracellular vesicles is/are substantially lower than the amount(s) of miR-10b and/or miR-10a in non-engineered extracellular vesicles.

Another aspect of the present invention provides a method of producing engineered extracellular vesicles, comprising genetically modifying a plurality/population of cells to overexpress miR-345 and let-7b, whereby the engineered extracellular vesicles produced by the cells comprise substantially greater amounts of miR-345 and let-7b than extracellular vesicles produced by cells not so genetically modified. Another aspect of the present invention provides a method of producing engineered extracellular vesicles, comprising loading a plurality/population of extracellular vesicles with miR-345 and let-7b, whereby the engineered extracellular vesicles comprise substantially greater amounts of miR-345 and let-7b than otherwise the same extracellular vesicles not so loaded. According to one embodiment of this aspect of the present invention, the amount(s) of miR-10b and/or miR-10a in the engineered extracellular vesicles is/are substantially lower than the amount(s) of miR-10b and/or miR-10a in non-engineered extracellular vesicles.

Another aspect of the present invention provides a method of producing engineered extracellular vesicles, comprising genetically modifying a plurality/population of cells to overexpress miR-345, miR-146, and let-7b, whereby the engineered extracellular vesicles produced by the cells comprise substantially greater amounts of miR-345, miR-146, and let-7b than extracellular vesicles produced by cells not so genetically modified. Another aspect of the present invention provides a method of producing engineered extracellular vesicles, comprising loading a plurality/population of extracellular vesicles with miR-345, miR-146, and let-7b, whereby the engineered extracellular vesicles comprise substantially greater amounts of miR-345, miR-146, and let-7b than otherwise the same extracellular vesicles not so loaded. According to one embodiment of this aspect of the present invention, the amount(s) of miR-10b and/or miR-10a in the engineered extracellular vesicles is/are substantially lower than the amount(s) of miR-10b and/or miR-10a in non-engineered extracellular vesicles.

Another aspect of the present invention provides a plurality/population of extracellular vesicles derived from a plurality/population of potent cells, wherein the amount of miR-345 in the extracellular vesicles derived from potent cells is substantially higher than the amount of miR-345 in extracellular vesicles derived from non-potent cells. According to one embodiment of this aspect of the present invention, the amount(s) of miR-10b and/or miR-10a in the extracellular vesicles derived from potent cells is/are substantially lower than the amount(s) of miR-10b and/or miR-10a in extracellular vesicles derived from non-potent cells.

Another aspect of the present invention provides a plurality/population of extracellular vesicles derived from a plurality/population of potent cells, wherein the amounts of miR-345 and miR-146a in the extracellular vesicles derived from potent cells are substantially higher than the amounts of miR-345 and miR-146a in extracellular vesicles derived from non-potent cells. According to one embodiment of this aspect of the present invention, the amount(s) of miR-10b and/or miR-10a in the extracellular vesicles derived from potent cells is/are substantially lower than the amount(s) of miR-10b and/or miR-10a in extracellular vesicles derived from non-potent cells.

Another aspect of the present invention provides a plurality/population of extracellular vesicles derived from a plurality/population of potent cells, wherein the amounts of miR-345 and let-7b in the extracellular vesicles derived from potent cells are substantially higher than the amounts of miR-345 and let-7b in extracellular vesicles derived from non-potent cells. According to one embodiment of this aspect of the present invention, the amount(s) of miR-10b and/or miR-10a in the extracellular vesicles derived from potent cells is/are substantially lower than the amount(s) of miR-10b and/or miR-10a in extracellular vesicles derived from non-potent cells.

Another aspect of the present invention provides a plurality/population of extracellular vesicles derived from a plurality/population of potent cells, wherein the amounts of miR-345, miR-146a, and let-7b in the extracellular vesicles derived from potent cells are substantially higher than the amounts of miR-345, miR-146a, and let-7b in extracellular vesicles derived from non-potent cells. According to one embodiment of this aspect of the present invention, the amount(s) of miR-10b and/or miR-10a in the extracellular vesicles derived from potent cells is/are substantially lower than the amount(s) of miR-10b and/or miR-10a in extracellular vesicles derived from non-potent cells.

Another aspect of the present invention provides a plurality of engineered cells produced by genetically modifying cells to overexpress miR-345. According to one embodiment of this aspect of the present invention, the amount(s) of miR-10b and/or miR-10a in the engineered cells is/are substantially lower than the amount(s) of miR-10b and/or miR-10a in cells not so genetically modified.

Another aspect of the present invention provides a plurality of engineered cells is produced by genetically modifying cells to overexpress miR-345 and miR-146a. According to one embodiment of this aspect of the present invention, the amount(s) of miR-10b and/or miR-10a in the engineered cell is/are substantially lower than the amount(s) of miR-10b and/or miR-10a in cells not so genetically modified.

Another aspect of the present invention provides a plurality of engineered cells produced by genetically modifying a cell to overexpress miR-345 and let-7b. According to one embodiment of this aspect of the present invention, the amount(s) of miR-10b and/or miR-10a in the engineered cells is/are substantially lower than the amount(s) of miR-10b and/or miR-10a in cells not so genetically modified.

Another aspect of the present invention provides a plurality of engineered cells produced by genetically modifying a cell to overexpress miR-345, miR-146a, and let-7b. According to one embodiment of this aspect of the present invention, the amount(s) of miR-10b and/or miR-10a in the engineered cells is/are substantially lower than the amount(s) of miR-10b and/or miR-10a in cells not so genetically modified.

According to one embodiment of the aforementioned aspects of the present invention, said genetic modification of cells is effected via transfection or transduction of one or more expression vectors encoding miR-345, miR-146a, and/or let-7b into the cells, and/or via CRISPR/Cas9 gene editing of the cells. According to one embodiment of the aforementioned aspects of the present invention, said expression vector comprises an expression control sequence. According to one embodiment of the aforementioned aspects of the present invention, said expression control sequence is a promoter, e.g., the cytomegalovirus (CMV) promoter.

According to one embodiment of the aforementioned aspects of the present invention, said loading of extracellular vesicles is effected with a chemical lipofection reagent or a chemical transfection reagent. According to one embodiment of the aforementioned aspects of the present invention, said chemical lipofection reagent or chemical transfection reagent is a polycationic lipid.

According to one embodiment of the aforementioned aspects of the present invention, the amounts of miR-345, miR-146a, and/or let-7b in the engineered extracellular vesicles are at least 2-fold, 3-fold, 4-fold, 5-fold, 6-fold, 7-fold, 8-fold, 9-fold, 10-fold, 15-fold, or 20-fold higher than the amounts of miR-345, miR-146a, and/or let-7b in non-engineered extracellular vesicles derived from the cells.

According to one embodiment of the aforementioned aspects of the present invention, the amount(s) of miR-10b and/or miR-10a in the engineered extracellular vesicles or the is engineered cells is/are at least 2-fold, 3-fold, 4-fold, 5-fold, 6-fold, 7-fold, 8-fold, 9-fold, 10-fold, 15-fold, or 20-fold lower than the amount(s) of miR-10b and/or miR-10a in non-engineered extracellular vesicles derived from the cells or otherwise the same non-engineered cells.

According to one embodiment of the aforementioned aspects of the present invention, said depletion of extracellular vesicles or cells of miR-10b and/or miR-10a is effected via CRISPR/Cas9 gene editing of the cells.

According to one embodiment of the aforementioned aspects of the present invention, the extracellular vesicles are exosomes, microvesicles, membrane particles, membrane vesicles, exosome-like vesicles, ectosomes, ectosome-like vesicles, exovesicles, epididimosomes, argosomes, promininosomes, prostasomes, dexosomes, texosomes, archeosomes, oncosomes, or the like.

According to one embodiment of the aforementioned aspects of the present invention, the cells are cardiosphere-derived cells (CDCs), explant-derived cells (EDCs) as described in, e.g., US/2012/0315252, mesenchymal stromal/stem cells (MSCs), or 293F cells. Additional non-limiting examples of the cells include newt A1 cells, fibroblasts such as normal human dermal fibroblasts (NHDFs), other stromal cells such as epithelial cells, endothelial cells, smooth muscle cells, keratinocytes, chondrocytes, neurons, glial cells, pericytes, and muscle satellite cells.

According to one embodiment of the aforementioned aspects of the present invention, the cells are immortalized. According to one embodiment of the aforementioned aspects of the present invention, the immortalized cells are produced by a method comprising: overexpressing simian virus 40 (SV40) small-t and large-T antigens in a culture of cells; and selecting a cell culture that can continue to double for at least 15 times. According to one embodiment of the aforementioned aspects of the present invention, the immortalized cells are produced by a method comprising: overexpressing c-Myc in a culture of cells; and selecting a cell culture that can continue to double for at least 15 times.

Another aspect of the present invention provides a method of treating an inflammatory disease or condition, or regenerating tissue in an individual having damaged tissue, in a subject in need thereof, the method comprising administering to the subject a therapeutically effective amount of the engineered extracellular vesicles according to the aforementioned aspects of the present invention, or a therapeutically effective amount of the extracellular vesicles derived from potent cells according to the aforementioned aspects of the is present invention. According to one embodiment of this aspect of the present invention, the damaged tissue comprises cardiac tissue or a skeletal muscle tissue. According to one embodiment of this aspect of the present invention, the inflammatory disease or condition is an inflammatory disease or condition with unbalanced macrophage response. According to one embodiment of this aspect of the present invention, the inflammatory disease or condition is a chronic inflammatory disease or condition. According to one embodiment of this aspect of the present invention, the chronic inflammatory disease or condition is macrophage activation syndrome, rheumatoid arthritis, inflammatory bowel disease, ulcerative colitis, psoriasis, or systemic lupus erythematosus. In several embodiments, the damaged tissue comprises one or more of neural and/or nervous tissue, epithelial tissue, skeletal muscle tissue, endocrine tissue, vascular tissue, smooth muscle tissue, liver tissue, pancreatic tissue, lung tissue, intestinal tissue, osseous tissue, connective tissue, or combinations thereof. In several embodiments, the damaged tissue is in need of repair, regeneration, or improved function due to an acute event. Acute events include, but are not limited to, trauma such as laceration, crush or impact injury, shock, loss of blood or oxygen flow, infection, chemical or heat exposure, poison or venom exposure, drug overuse or overexposure, and the like. For example, in several embodiments, the damaged tissue is cardiac tissue and the acute event comprises a myocardial infarction. In some embodiments, administration of a therapeutically effective amount of the engineered extracellular vesicles according to the aforementioned aspects of the present invention, or a therapeutically effective amount of the extracellular vesicles derived from potent cells according to the aforementioned aspects of the present invention, results in an increase in cardiac wall thickness in the area subjected to the infarction. In additional embodiments, the tissue is damaged due to chronic disease or ongoing injury. For example, progressive degenerative diseases can lead to tissue damage that propagates over time (at times, even in view of attempted therapy). Chronic disease need not be degenerative to continue to generate damaged tissue, however. In several embodiments, chronic disease/injury includes, but it not limited to epilepsy, Alzheimer's disease, Parkinson's disease, Huntington's disease, dopaminergic impairment, dementia, ischemia including focal cerebral ischemia, ensuing effects from physical trauma (e.g., crush or compression injury in the CNS), neuro degeneration, immune hyperactivity or deficiency, bone marrow replacement or functional supplementation, arthritis, auto-immune disorders, inflammatory bowel disease, cancer, diabetes, muscle weakness (e.g., muscular dystrophy, amyotrophic lateral sclerosis, and the like), blindness and hearing loss. Cardiac tissue, in several embodiments, is also subject to damage due to chronic disease, such as for example is congestive heart failure, ischemic heart disease, diabetes, valvular heart disease, dilated cardiomyopathy, infection, and the like. Other sources of damage also include, but are not limited to, injury, age-related degeneration, cancer, and infection.

Another aspect of the present invention provides an assay method comprising: measuring the amount(s) of miR-345 and/or miR-10b in extracellular vesicles produced by a cell line; and correlating the measure with the potency of the extracellular vesicles, wherein the amount of miR-345 is positively associated with potency and the amount of miR-10b is negatively associated with potency. According to one embodiment of this aspect of the present invention, the method further comprises: measuring the amount(s) of miR-146a and/or let 7b; and correlating the amounts with the potency of the extracellular vesicles, wherein the amount(s) of miR-146a and let-7b are positively associated with potency. Another aspect of the present invention provides a method comprising administering to a subject an extracellular vesicle composition determined to be potent by the method provided by the aforementioned aspect of the present invention. According to one embodiment of these aspects of the present invention, an extracellular vesicle composition is potent if it increases the left ventricular ejection fraction in a mouse model of myocardial infarction. According to one embodiment of these aspects of the present invention, the extracellular vesicles are exosomes, microvesicles, membrane particles, membrane vesicles, exosome-like vesicles, ectosomes, ectosome-like vesicles, exovesicles, epididimosomes, argosomes, promininosomes, prostasomes, dexosomes, texosomes, archeosomes, oncosomes, or the like. Another aspect of the present invention provides an extracellular vesicle composition determined to be potent by the method provided by the aforementioned aspect of the present invention, and a pharmaceutically acceptable carrier.

Another aspect of the present invention provides a kit for performing the assay method provided by the aforementioned aspect of the present invention comprising: a means for measuring the amount(s) of miR-345 and/or miR-10b in extracellular vesicles produced by a cell line. According to one embodiment of this aspect of the present invention, the kit further comprises a means for measuring the amount(s) of miR-146a and/or let 7b.

Another aspect of the present invention provides a method of producing a plurality of engineered extracellular vesicles, comprising culturing a plurality of cells genetically modified to overexpress miR-345, miR-146, and/or let-7b, and/or decrease the expression of miR-10b, wherein the cells produce extracellular vesicles comprising increased amounts of miR-345, miR-146 and/or let-7b, and/or a decreased amount of miR-10b, to produce conditioned media; and harvesting the extracellular vesicles from the conditioned media. According to one embodiment of this aspect of the present invention, the cells are cultured in serum free media. According to one embodiment of this aspect of the present invention, the extracellular vesicles are exosomes, microvesicles, membrane particles, membrane vesicles, exosome-like vesicles, ectosomes, ectosome-like vesicles, exovesicles, epididimosomes, argosomes, promininosomes, prostasomes, dexosomes, texosomes, archeosomes, oncosomes, or the like. According to one embodiment of this aspect of the present invention, the cells are cardiosphere-derived cells (CDCs), explant-derived cells (EDCs) as described in, e.g., US/2012/0315252, mesenchymal stromal/stem cells (MSCs), or 293F cells. Additional non-limiting examples of the cells include newt A1 cells, fibroblasts such as normal human dermal fibroblasts (NHDFs), other stromal cells such as epithelial cells, endothelial cells, smooth muscle cells, keratinocytes, chondrocytes, neurons, glial cells, pericytes, and muscle satellite cells.

Another aspect of the present invention provides a method comprising: producing a plurality compositions of extracellular vesicles from each of a plurality of different cell lines; measuring the amounts of miR-345, miR-146a, let-7b, and/or miR-10b in the extracellular vesicle compositions; and correlating the amounts with potency of the extracellular vesicle compositions, wherein increased amounts of miR-345, miR-146, and/or let-7b are positively associated with potency, and a deceased amount of miR-10b is positively associated with potency; and selecting potent extracellular vesicle compositions.

### BRIEF DESCRIPTION OF THE DRAWINGS

FIG. 1. Demographic characteristics of CDC and MSC donors. Ad-MSC, adipose-derived mesenchymal stem cell; BM-MSC, bone marrow-derived mesenchymal stem cell; F, female; M, male.

FIG. 2. Schematic overview of the steps involved to isolate CDC- and MSC-derived EVs. To isolate CDC- or MSC-derived EVs, cells were grown till confluency at passage 5. The cells were thoroughly washed and fed with serum-free media. Next, the cells were incubated for 48 hours or 15 days prior to collection, filtered with a 0.45-μm filter, and stored at −80° C. EVs were isolated using 10 kDa MWCO ultrafiltration filters (ultrafiltration by centrifugation, UFC).

FIG. 3. Representative nanoparticle tracking analysis (NS300, Nanosight) showing particle size distribution and concentration for MSC-EVs and CDC-EVs.

FIG. 4. Exosomes isolated from CDCs visualized by transmission electron microscopy.

FIG. 5. The mode particle size was determined by nanoparticle tracking analysis for CDC-EVs and MSC-EVs with a significant increase in particle size of CDC-EVs compared to MSC-EVs. Results are mean±SEM, Mann-Whitney test, *P<0.05; CDC-EVs, n=18; MSC-EVs, n=3.

FIG. 6. Particle concentration (particles/mL) was determined with a significant higher concentration of CDC-EVs compared to MSC-EVs. Results are mean±SEM, Mann-Whitney test, *P<0.005; CDC-EVs, n=18; MSC-EVs, n=3.

FIG. 7. The protein concentration (mg/mL) in CDC-EVs was significantly higher than in MSC-EVs. Results are mean±SEM, Mann-Whitney test, *P<0.05; CDC-EVs, n=22; MSC-EVs, n=4.

FIG. 8. Protein immunoblots of exosome markers in CDC-EVs.

FIG. 9. The colocalization and relative distribution of surface markers CD81, CD63 and CD9 on CDC-EVs and MSC-EVs was analyzed using NanoView Biosciences technology.

FIG. 10. CDC-EVs (n=7) were profiled for various surface markers using the MACSPlex exosome kit.

FIG. 11. The Y RNA and miRNA composition of 15 days CDC-EVs (n=5) and MSC-EVs (n=3) and 48 hours MSC-EVs (n=3) showed differences with the lowest Y RNA and miRNA content in 15 days and 48 hours MSC-EVs compared to 15 days CDC-EVs.

FIG. 12. hY4 was relatively the most abundant Y RNA species in both CDC-EVs and MSC-EVs compared to hY1, hY3 and hY5 with a significant increase in CDC-EVs. hY5 was relatively significantly more abundant in MSC-EVs compared to CDC-EVs. Results are mean±SEM, Holm-Sidak method, *P<0.05; CDC-EVs, n=5; MSC-EVs, n=3.

FIG. 13. Differential K-means clustering of CDC-EVs (n=12) compared to MSC-EVs (n=4) of uniquely-alignable reads conforming miRNAs. For each sample pair, the color code indicates the percentage of downsampling trials where the two samples are grouped into the same cluster.

FIG. 14. Schematic overview of the in vivo MI mouse model to test the potency of different CDC donors. A permanent ligation of the LAD was performed to induce an MI in SCID beige mice, immediately followed by intramyocardial injection of CDCs (100,000) or placebo (PBS) in the border zone. One day post-MI, echocardiographic measurements were performed to determine the ejection fraction (EF) which was repeated at day 21. A CDC cell line derived from a donor is designated as potent when an improvement of the delta EF compared to placebo is observed.

FIG. 15. Left ventricular ejection fraction (LVEF), measured by echocardiography, one day after permanent LAD ligation (baseline) and after 21 days. Results are mean±SEM, MannWhitney test, *P<0.01; PBS, n=5; non-potent CDCs, n=7; potent CDCs, n=9.

FIG. 16. Schematic overview of the in vitro peritoneal macrophage assay. Mice or rats were i.p. injected with 3% Brewer's thioglycollate media, 3 days prior to peritoneal lavage to isolate the peritoneal macrophages. Macrophages were plated and treated with CDC- or MSCEVs. After 6 hours, macrophages were harvested and gene expression of Arg1 and Nos2 was analyzed.

FIG. 17. The average fold mouse Arg1/Nos2 expression of potent CDC-EVs (n=5) was significantly increased compared to non-potent CDC-EVs (n=5; #, P<0.01) and no treatment (n=5; #, P<0.01). Treatment with MSC-EVs (n=3) showed an increased Arg1/Nos2 expression compared to NT (*, P<0.05). Results depict mean±SEM and fold expression compared to NT, Kruskal-Wallis test, *P<0.05, #P<0.01.

FIG. 18. The average fold rat Arg1/Nos2 expression of 4 potent CDC-EVs (dose of 2500 particles per cell) was significantly increased compared to 3 non-potent CDC-EVs (dose of 500 and 2500 particles per cell) and in addition to the 4 potent CDC-EVs dosed with 500 particles per cell. Results depict mean±SEM fold expression compared to NT (dotted line), one-way ANOVA analysis, *P<0.0001 compared to potent 2500p.

FIG. 19. Schematic overview of the in vivo peritonitis mouse model. Mice were i.p. injected with 100 μg zymosan together with tail vein injection of placebo (P) or CDC-EVs (E) (1.5-3×10¹⁰ particles/200 μL/mouse). On day 2, mice received a second dose of placebo (PP, EP) or CDC-EVs (EE) via tail vein injection. After 3 days, the peritoneal macrophages were isolated through peritoneal lavage and the accumulation of activated macrophages was analyzed by flow cytometry.

FIG. 20. The average number of CD11b⁺F4/80⁺ was significantly lower in mice treated with potent CDC-EVs (n=6) compared to non-potent CDC-EVs (n=3) with the highest reduction in mice treated with 2 doses of potent CDC-EVs (EE). Results depict mean±SEM and relative compared to PP (dotted line), one-way ANOVA analysis, *P<0.05.

FIG. 21. Potent (n=6) versus non-potent (n=5) CDC-EVs showed a differential K-means clustering of uniquely-alignable reads conforming miRNAs. For each sample pair, the color code indicates the percentage of downsampling trials where the two samples are grouped into the same cluster.

FIG. 22. The miRNA analysis of potent (n=6) versus non-potent (n=5) CDC-EVs showed a significantly increased expression of miR-345-5p in potent compared to non-potent CDC-EVs with an FDR of 9e−4.

FIG. 23. Confirmation by qPCR of upregulated expression of miR-345-5p in potent CDC-EVs compared to non-potent CDC-EVs and MSC-EVs. Results depict mean±SEM and fold expression compared to MSC-EV (dotted line), Mann-Whitney test, *P<0.001. MSC-EVs, n=5; non-potent CDC-EVs, n=6; potent CDC-EVs, n=20.

FIG. 24. Macrophages transfected with miR-345-5p mimic showed a higher Arg1:Nos2 ratio after 6 hours but not after 24 hours compared to macrophages transfected with miR-scramble.

FIG. 25. Protein levels of the miR-345-5p target RelA, were decreased after 6 hours but not 24 hours compared to macrophages transfected with miR-scramble.

FIG. 26 and FIG. 27. The miRNA analysis of potent (n=6) versus non-potent (n=5) CDC-EVs showed a significantly increased expression of let-7b-5p and decreased expression of miR-151a-3p in potent compared to non-potent CDC-EVs with an FDR of respectively 5e-2 and 8e-2.

FIG. 28. Confirmation by qPCR of upregulated expression of let-7b-5p in potent CDCEVs compared to non-potent CDC-EVs and MSC-EVs. Results depict mean±SEM and fold expression compared to MSC-EV (dotted line), Mann-Whitney test, *P<0.05. MSC-EVs, n=5; non-potent CDC-EVs, n=6; potent CDC-EVs, n=20.

FIG. 29. Patient demographics for each cell donor. Ad-MSC adipose-derived mesenchymal stem cell, BM-MSC bone marrow-derived mesenchymal stem cell, CDC cardiosphere-derived cells, F female, M male.

FIG. 30. Relative differences in protein surface marker expression between CDC-EVs (n=8, 15 days serum-free media) and MSC-EVs (n=4, 15 days serum-free media).

FIG. 31. miRNA analysis of CDC-EVs and MSC-EVs revealed a significant increase in expression of miR-10b-5p in MSC-EVs compared to CDC-EVs.

FIG. 32. Quantitative qPCR analysis of miR-10b in EVs. Results are presented as mean±SEM. CDC-EVs (n=10); MSC-EVs (n=4). Statistical significance was determine using the Mann-Whitney test, *P<0.05.

FIG. 33. Schematic overview of the in vivo MI mouse model.

FIG. 34. Percent change in ejection fraction (ΔEF) between days 28 and 1 post-MI. Results are depicted as mean±SEM. Statistical significance was determined using 1-way ANOVA followed by Tukey's multiple comparisons test. *P<0.05.

FIG. 35. Representative images of Masson's trichrome staining.

FIG. 36. Quantitative analysis of scar size in FIG. 35. Results are depicted as mean f SEM. Statistical significance was determined using 1-way ANOVA followed by Tukey's multiple comparisons test. *P<0.05.

FIG. 37. Quantitative analysis of infarct wall thickness (IWT) in FIG. 35. Results are depicted as mean±SEM. Statistical significance was determined using 1-way ANOVA followed by Tukey's multiple comparisons test. *P<0.05.

FIG. 38. Gene expression of in vitro plated thioglycolate-stimulated peritoneal macrophages treated with or without EVs. NT no treatment. Results are depicted as mean f SEM. Statistical significance was determined using 1-way ANOVA followed by Tukey's multiple comparisons test. *P<0.05.

FIG. 39. Gene expression of in vitro plated thioglycolate-stimulated peritoneal macrophages treated with miR-10b mimic or miR scrambled control. Results are depicted as mean±SEM. Statistical significance was determined using 1-way ANOVA followed by Tukey's multiple comparisons test. *P<0.05.

FIG. 40. Schematic overview of the acute peritonitis mouse model. Mice received an intraperitoneal (i.p.) injection of zymosan (day 0) and then intravenous (i.v.) delivery of placebo (P) or EVs (E) (days 0 and 1). Animals were sacrificed on day 2 and peritoneal exudate collected for flow cytometry.

FIG. 41. Representative flow plots of peritoneal cells collected on day 2.

FIG. 42. Quantification of CD11b+F4/80+ cells in FIG. 40. Results are depicted as mean±SEM. Statistical significance was determined using 1-way ANOVA followed by Tukey's multiple comparisons test. *P<0.05.

FIG. 43. Number of miR-146a reads in MSC-EVs and CDC-EVs. *P<0.05.

FIG. 44. Dose-dependent relative gene expression of Arg1 and Nos2 following CDC-EV treatment. Two EV doses were tested: 500 and 2500 particles/cell. Untreated control macrophages are depicted by the dashed line. *P<0.05.

### DETAILED DESCRIPTION OF THE INVENTION

### A. Definitions

The sequence of miR-345 is
(SEQ ID NO: 1)
ACCCAAACCCUAGGUCUGCUGACUCCUAGUCCAGGGCUCGUGAUGGCUG
GUGGGCCCUGAACGAGGGGUCUGGAGGCCUGGGUUUGAAUAUCGACAGC.
The sequence of miR-345-5p (18-39) is
(SEQ ID NO: 2)
GCUGACUCCUAGUCCAGGGCUC.
The sequence of miR-345-3p (54-75) is
(SEQ ID NO: 3)
GCCCUGAACGAGGGGUCUGGAG.
The sequence of miR-146a is
(SEQ ID NO: 4)
CCGAUGUGUAUCCUCAGCUUUGAGAACUGAAUUCCAUGGGUUGUGUCAG
UGUCAGACCUCUGAAAUUCAGUUCUUCAGCUGGGAUAUCUCUGUCAUCG
U.
The sequence of miR-146a-5p (21-42) is
(SEQ ID NO: 5)
UGAGAACUGAAUUCCAUGGGUU.
The sequence of miR-146a-3p (57-78) is
(SEQ ID NO: 6)
CCUCUGAAAUUCAGUUCUUCAG.
The sequence of let-7b is
(SEQ ID NO: 7)
CGGGGUGAGGUAGUAGGUUGUGUGGUUUCAGGGCAGUGAUGUUGCCCCU
CGGAAGAUAACUAUACAACCUACUGCCUUCCCUG.
The sequence of let-7b-5p (6-27) is
(SEQ ID NO: 8)
UGAGGUAGUAGGUUGUGUGGUU
The sequence of let-7b-3p (60-81) is
(SEQ ID NO: 9)
CUAUACAACCUACUGCCUUCCC.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

As used herein, “and/or” refers to and encompasses any and all possible combinations of one or more of the associated listed items, as well as the lack of combinations when interpreted in the alternative (or).

The terms “about” and “approximate”, as used herein when referring to a measurable value such as an amount, dose, time, temperature, activity, level, number, frequency, percentage, dimension, size, amount, weight, position, length and the like, is meant to encompass variations of ±15%, ±10%, ±5%, ±1%, ±0.5%, or even ±0.1% of the is specified amount, dose, time, temperature, activity, level, number, frequency, percentage, dimension, size, amount, weight, position, length and the like.

The term “derived from” as in “A is derived from B” means that A is obtained from B in such a manner that A is not identical to B.

The terms “treat”, “therapeutic”, “prophylactic” and “prevent” are not intended to be absolute terms. Treatment, prevention and prophylaxis can refer to any delay in onset, amelioration of symptoms, improvement in patient survival, increase in survival time or rate, etc. Treatment, prevention, and prophylaxis can be complete or partial. The term “prophylactic” means not only “prevent”, but also minimize illness and disease. For example, a “prophylactic” agent can be administered to subject to prevent infection, or to minimize the extent of illness and disease caused by such infection. The effect of treatment can be compared to an individual or pool of individuals not receiving the treatment, or to the same patient prior to treatment or at a different time during treatment. In some aspects, the severity of disease is reduced by at least 10%, as compared, e.g., to the individual before administration or to a control individual not undergoing treatment. In some aspects, the severity of disease is reduced by at least 25%, 50%, 75%, 80%, or 90%, or in some cases, no longer detectable using standard diagnostic techniques.

A treatment can be considered “effective,” as used herein, if one or more of the signs or symptoms of a condition described herein are altered in a beneficial manner, other clinically accepted symptoms are improved, or even ameliorated, or a desired response is induced e.g., by at least 2%, 3%, 4%, 5%, 10%, or more, following treatment according to the methods described herein. Efficacy can be assessed, for example, by measuring a marker, indicator, symptom, and/or the incidence of a condition treated according to the methods described herein or any other measurable parameter appropriate. Efficacy can also be measured by a failure of an individual to worsen as assessed by hospitalization, or need for medical interventions (e.g., progression of the disease is halted). Treatment includes any treatment of a disease in an individual or an animal (some non-limiting examples include a human or an animal) and includes: (1) inhibiting the disease, e.g., preventing a worsening of symptoms (e.g. pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response. One is skilled in the art can monitor efficacy of administration and/or treatment by measuring any one of such parameters, or any combination of parameters.

The term “effective amount” as used herein refers to the amount of a composition or an agent needed to alleviate at least one or more symptom of the disease or disorder, and relates to a sufficient amount of therapeutic composition to provide the desired effect. The term “therapeutically effective amount” refers to an amount of a composition or therapeutic agent that is sufficient to provide a particular effect when administered to atypical subject. An effective amount as used herein, in various contexts, can include an amount sufficient to delay the development of a symptom of the disease, alter the course of a symptom disease (for example but not limited to, slowing the progression of a symptom of the disease), or reverse a symptom of the disease. For example, for the given parameter, a therapeutically effective amount will show an increase or decrease of therapeutic effect at least any of 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least any of a 1.2-fold, 1.5-fold, 2-fold, 5-fold, or more effect over a control. The therapeutically effective amount may be administered in one or more doses of the therapeutic agent. The therapeutically effective amount may be administered in a single administration, or over a period of time in a plurality of doses.

“Administering” as used herein can include any suitable routes of administering a therapeutic agent or composition as disclosed herein. Suitable routes of administration include, without limitation, oral, parenteral, intravenous, intramuscular, subcutaneous, transdermal, airway (aerosol), pulmonary, cutaneous, injection or topical administration. Administration can be local or systemic.

As used herein, the term “pharmaceutically acceptable” refers to a carrier that is compatible with the other ingredients of the formulation and not deleterious to the recipient thereof. The term is used synonymously with “physiologically acceptable” and “pharmacologically acceptable”. A pharmaceutical composition will generally comprise agents for buffering and preservation in storage, and can include buffers and carriers for appropriate delivery, depending on the route of administration. The phrase “pharmaceutically acceptable” is employed herein to refer to those compounds, materials, compositions, and/or dosage forms which are, within the scope of sound medical judgment, suitable for use in contact with the tissues of human beings and animals without excessive is toxicity, irritation, allergic response, or other problem or complication, commensurate with a reasonable benefit/risk ratio.

The terms “dose” and “dosage” are used interchangeably herein. A dose refers to the amount of active ingredient given to an individual at each administration. For the present invention, the dose can refer to the concentration of the extracellular vesicles or associated components, e.g., the amount of therapeutic agent or dosage of radiolabel. The dose will vary depending on a number of factors, including frequency of administration; size and tolerance of the individual; severity of the condition; risk of side effects; the route of administration; and the imaging modality of the detectable moiety (if present). One of skill in the art will recognize that the dose can be modified depending on the above factors or based on therapeutic progress. The term “dosage form” refers to the particular format of the pharmaceutical, and depends on the route of administration. For example, a dosage form can be in a liquid, e.g., a saline solution for injection.

“Subject,” “patient,” “individual” and like terms are used interchangeably and refer to, except where indicated, mammals such as humans and non-human primates, as well as rabbits, rats, mice, goats, pigs, and other mammalian species. The term does not necessarily indicate that the subject has been diagnosed with a particular disease, but typically refers to an individual under medical supervision. A patient can be an individual that is seeking treatment, monitoring, adjustment or modification of an existing therapeutic regimen, etc.

As used herein, the following meanings apply unless otherwise specified. The word “may” is used in a permissive sense (i.e., meaning having the potential to), rather than the mandatory sense (i.e., meaning must). The words “include”, “including”, and “includes” and the like mean including, but not limited to. The singular forms “a,” “an,” and “the” include plural referents. Thus, for example, reference to “an element” includes a combination of two or more elements, notwithstanding use of other terms and phrases for one or more elements, such as “one or more.” The term “or” is, unless indicated otherwise, non-exclusive, i.e., encompassing both “and” and “or.” The term “any of” between a modifier and a sequence means that the modifier modifies each member of the sequence. So, for example, the phrase “at least any of 1, 2 or 3” means “at least 1, at least 2 or at least 3”. The phrase “at least one” includes “a plurality”.

Definitions of common terms in cell biology and molecular biology can be found in “The Merck Manual of Diagnosis and Therapy”, 19th Edition, published by Merck Research Laboratories, 2006 (ISBN 0-91 1910-19-0); Robert S. Porter et al. (eds.), The Encyclopedia is of Molecular Biology, published by Blackwell Science Ltd., 1994 (ISBN 0-632-02182-9); Benjamin Lewin, Genes X, published by Jones & Bartlett Publishing, 2009 (ISBN-10: 0763766321); Kendrew et al. (eds.), Molecular Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al., eds.

A variety of host cells are known in the art and suitable for proteins expression and extracellular vesicles production. Non-limiting examples of typical cell used for transfection include, but are not limited to, a bacterial cell, a eukaryotic cell, a yeast cell, an insect cell, or a plant cell. For example, human embryonic kidney 293 (HEK293), E. coli, Bacillus, Streptomyces, Pichia pastoris, Salmonella typhimurium, Drosophila S2, Spodoptera SJ9, CHO, COS (e.g. COS-7), 3T3-F442A, HeLa, HUVEC, HUAEC, NIH 3T3, Jurkat, 293, 293H, or 293F.

The term “pharmaceutical composition” refers to a formulation comprising an active ingredient, and optionally a pharmaceutically acceptable carrier, diluent or excipient. The term “active ingredient” can interchangeably refer to an “effective ingredient,” and is meant to refer to any agent that is capable of inducing a sought-after effect upon administration. By “pharmaceutically acceptable” it is meant the carrier, diluent or excipient must be compatible with the other ingredients of the formulation and not deleterious to the recipient thereof, nor to the activity of the active ingredient of the formulation. Pharmaceutically acceptable carriers, excipients or stabilizers are well known in the art, for example Remington's Pharmaceutical Sciences, 16th edition, Osol, A. Ed. (1980). Pharmaceutically acceptable carriers, excipients, or stabilizers are nontoxic to recipients at the dosages and concentrations employed, and may include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or is sorbitol; salt-forming counter-ions such as sodium; metal complexes (for example, Zn-protein complexes); and/or non-ionic surfactants such as TWEEN™, PLURONICS™ or polyethylene glycol (PEG). Examples of carrier include, but are not limited to, liposome, nanoparticles, ointment, micelles, microsphere, microparticle, cream, emulsion, and gel. Examples of excipient include, but are not limited to, anti-adherents such as magnesium stearate, binders such as saccharides and their derivatives (sucrose, lactose, starches, cellulose, sugar alcohols and the like) protein like gelatin and synthetic polymers, lubricants such as talc and silica, and preservatives such as antioxidants, vitamin A, vitamin E, vitamin C, retinyl palmitate, selenium, cysteine, methionine, citric acid, sodium sulfate and parabens. Examples of diluent include, but are not limited to, water, alcohol, saline solution, glycol, mineral oil and dimethyl sulfoxide (DMSO).

As a non-limiting example, what is meant by “potent” or “sufficiently potent” cells according to the present invention is that such cells are capable of improving a particular disease state by an appreciable degree as measured by a mouse model of acute myocardial infarction. For instance, administration of “potent” cells in the heart of an infarcted mouse would increase the left ventricular ejection fraction (ΔLVEF >0%), more preferably by at least 2% (ΔLVEF ≥2%), and more preferably by at least 4% (ΔLVEF ≥4%) at day 21 compared to day 1. See, e.g., Smith et al., Regenerative potential of cardiosphere-derived cells expanded from percutaneous endomyocardial biopsy specimens, Circulation. 2007 Feb. 20; 115(7):896-908.

What is meant by “non-potent” cells according to the present invention is that such cells are incapable of improving a particular disease state. For instance, administration of “non-potent” cells in infarcted mice would lead to a change in ejection fraction similar to non-treated animals (ΔLVEF ≤0%). Id.

The term “chemical lipofection reagent” or “chemical transfection reagent” refers to a cationic-lipid transfection reagent, e.g., Lipofectamine® MessengerMAX™, Lipofectamine® 2000, Lipofectamine® 3000, used to increase the transfection efficiency of RNA (including miRNA) or plasmid DNA into in vitro cell cultures.

As used herein the term an “engineered” extracellular vesicle refers to an extracellular vesicle with an internal luminal space exogenously modified in its composition. As used herein the term “exogenously modified” has the plain and ordinary meaning in the field of cell therapy, i.e., increasing the level or concentration of, e.g., a microRNA of interest in a is cell by the action of a molecular factor that originates from outside the cell. An engineered extracellular vesicle can be exogenously modified by a chemical, a physical, or a biological method, or by being produced from a cell previously modified by such exogenous modification. Non-limiting examples include introduction of transient or stable genetic material that increases the availability of, e.g., a microRNA of interest. Non-limiting examples include transfection by plasmids or other genetic material or the use of viral vectors. In contrast, merely selecting a cell, or a group of cells, based on a preexisting or native high level of a particular nucleic acid, a protein, a small molecule, a lipid, a carbohydrate, etc. of interest would not be “exogenously” increasing the level thereof.

As used herein the term an “engineered” cell refers to a cell with its intracellular composition exogenously modified. As used herein the term “exogenously modified” has the plain and ordinary meaning in the field of cell therapy, i.e., increasing the level or concentration of, e.g., a microRNA of interest in a cell by the action of a molecular factor that originates from outside the cell. An engineered cell can be exogenously modified by a chemical, a physical, or a biological method. Non-limiting examples include introduction of transient or stable genetic material that increases the availability of, e.g., a microRNA of interest. Non-limiting examples include transfection by plasmids or other genetic material or the use of viral vectors. In contrast, merely selecting a cell, or a group of cells, based on a preexisting or native high level of a particular nucleic acid, a protein, a small molecule, a lipid, a carbohydrate, etc. of interest would not be “exogenously” increasing the level thereof.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or is to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

### B. Introduction

Cardiosphere-derived cells (CDC) are heart derived cells that possess cardioprotective, regenerative and immunomodulatory characteristics (1-3). It has been previously shown that most of the beneficial effects mediated by the administration of CDCs can be replicated by CDC-derived extracellular vesicles (CDC-EVs) and in fact, abolishing the ability of CDCs to secrete EVs prevents their therapeutic effects (4-6). Extracellular vesicles are lipid bilayer nanoparticles secreted by almost all cell types and that function as intercellular communication tools. EVs contain specific proteins (transcription factors, transmembrane receptors, integrins, etc.), lipids and nucleic acids (miRNA, mRNA, Y RNA, etc.) that once are released into the target cells, alter their function and behavior (7, 8).

After a cardiac ischemic event, an inflammatory response occurs that leads to healing is and scar formation. In the acute phase, necrosis elicits activation of the complement system and secretion of various pro-inflammatory cytokines followed by neutrophil and macrophage recruitment. Neutrophils and macrophages are attracted to clear necrotic debris. Next, cytokines and growth factors are released leading to angiogenesis, fibroblast proliferation and extracellular matrix production. Finally, the inflammation resolves, and healing scar tissue is formed. Macrophages play a crucial role in this process since infiltrating monocytes differentiate into pro-inflammatory M1 macrophages soon after the insult and later on during the healing phase, when M2 anti-inflammatory macrophages are recruited (9-12). It has been shown that CDCs and CDC-EVs polarize macrophages into a healing phenotype that leads to modulation of the inflammatory response and to promotion of tissue regeneration in in vivo animal models of ischemic injury (1, 13). These immunomodulatory capabilities seem not to be restricted to CDC-EVs and have also been described when mesenchymal stem cell (MSC) extracellular vesicles (MSC-EVs) were used (14-17). The ability of CDC-EVs to reduce the inflammatory reaction by macrophages supports the therapeutic utility of CDC-EVs in a range of inflammatory diseases.

CDCs derived from different donors possess variable regenerative potency in an MI animal model (18, 19). A CDC donor is considered potent when an improvement significantly different from placebo treated mice in left ventricular ejection fraction (LVEF) is observed 3 weeks post-MI. Previous attempts to identify donor characteristics able to predict CDCs potency did not render clear results. No significant regenerative potency in this model is observed when MSCs are administered. Understanding what determines potency is critical for the design of a manufacturing process able to produce equivalent products with comparable bioactivity.

For the first time the present inventors performed a direct comparison of the small RNA composition and immunomodulatory bioactivity of EVs derived from MSC and from CDCs. The present inventors have also studied small RNA profile and bioactivity of EVs from CDCs with different regenerative capabilities.

In the present specification, the present inventors showed that EVs from potent CDCs polarize macrophages toward an anti-inflammatory phenotype and reduce macrophage recruitment in vivo in a sepsis mouse model. In addition, the present inventors showed that CDC-EVs contain a unique RNA cargo set that can differentiate them from MSC-EVs and a signature of 3 miRNAs that are differentially expressed in EVs derived from potent and non-potent CDCs.

The identification of miRNAs able to discriminate between EVs with different regenerative capabilities paves the way to engineer cellular products to produce EVs with superior capabilities.

### C. Isolation and Culture of Human CDCs

Donor hearts were obtained from organ procurement organizations under an IRB-approved protocol and processed as described by R R Makkar et al. (20) with modifications. A combination of atrial and septal tissue was used to seed explant fragments without previous collagenase digestion. Explants were seeded onto CellBIND surface culture flasks (Corning) for 10-21 days before harvest of explant-derived cells (EDCs) and formation of cardiopsheres in ultra-low attachment surface flasks (Corning) for 3 days. CDCs were obtained by seeding cardiospheres onto fibronectin-coated dishes and passaged till passage 5. All cultures were maintained at 5% CO₂, 5% O₂ at 37° C., using IMDM (GIBCO; supplemented with 20% bovine serum (Equafetal, Atlas), 0.5 μg/mL gentamycin, and 99 μM 2-mercaptoethanol).

### D. Generation and Purification of EVs

CDC-EVs and MSC-EVs were generated from confluent CDCs or MSCs, respectively at passage 5. Confluent cells at passage 5 were thoroughly washed with IMDM with phenol red before addition of serum-free IMDM without phenol red medium. After 48 hours (MSCs) or 15 days (CDCs and MSCs) of culture, conditioned medium was collected and purified with a 0.45-μm filter to remove cellular debris and aggregates prior to storage at −80° C. At time of concentration, conditioned medium was thawed at 37° C. and concentrated using ultrafiltration by centrifugation (UFC). A 10 kDa centrifugation filter was applied (Amicon, Millipore) according manufacturer's protocol. Next, the retentate (UFC fraction) containing EVs was characterized using nanoparticle tracking technology (NS300, Nanosight) to determine particle size, and concentration and protein concentration was assessed using the DC protein assay (Bio-Rad) according to manufacturer's protocol.

### E. Characterization of CDC- and MSC-Derived EVs

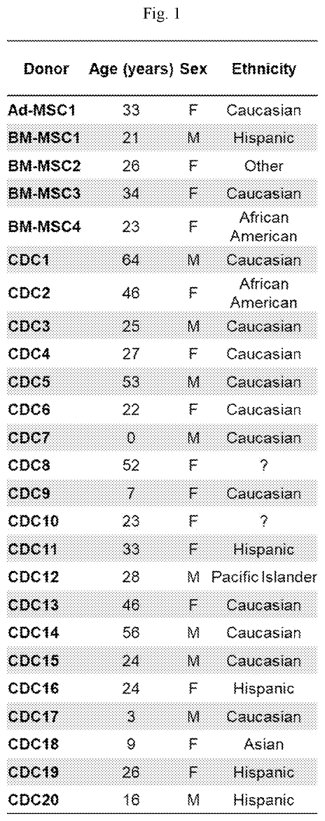

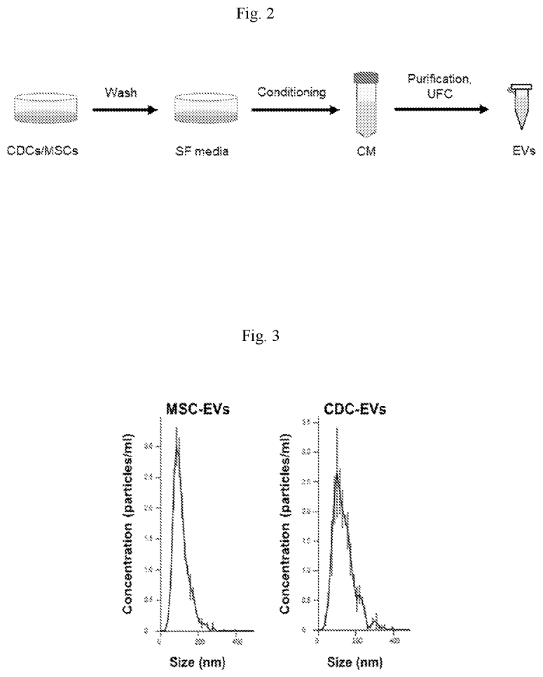

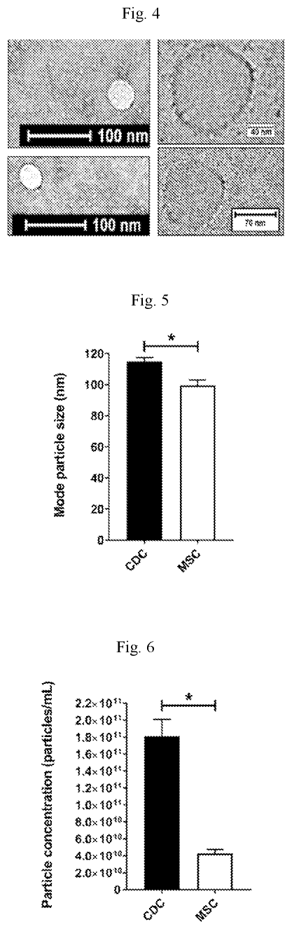

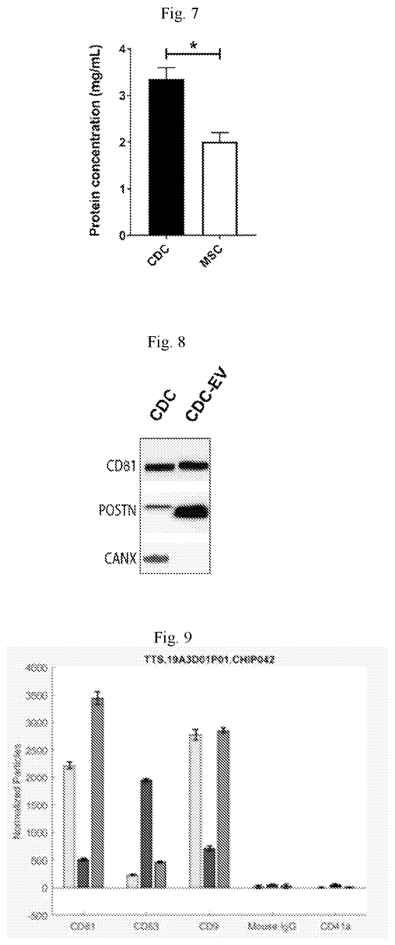

CDCs from 20 different donors (FIG. 1) were isolated and in vitro expanded as previously described (20). MSCs from 4 different donors were obtained from Lonza and expanded according to the manufacturer's recommendations. CDC- and MSC-EVs were isolated from 15 days serum-free media collected from confluent passage 5 (P5) cells (FIG. 2). In addition, EVs from MSCs cultured for 48 hours in serum-free media were isolated as this approach is usually described in the literature for MSC-EV collection. The conditioned media was concentrated by ultrafiltration by centrifugation (UFC) using 10 kDa MWCO filters followed by nanoparticle tracking analysis (Nanosight) (FIG. 3). The presence of EVs in conditioned media was confirmed through electron microscopy (FIG. 4). The Nanosight readings provide the particle size (FIG. 5) and concentration (FIG. 6). CDC-EVs (n=18, 114.9±2.5 nm) showed a significant increased mode particle size in diameter compared to MSC-EVs (n=3, 99.7±3.3) (P<0.05); however, both cell type-derived EVs showed values within the typical EV range (7, 13, 21). The particle concentration of CDC-EVs (n=18, 2.8e11±0.9e11 particles/mL) was significantly higher compared to MSC-EVs (n=3, 4.3e10±0.3e10 particles/mL) (P<0.005). In addition, the present inventors also observed a significant higher protein concentration in CDC-EVs (3.5±0.2 mg/mL) compared to MSC-EVs (2.0±0.2 mg/mL) (P<0.05) (FIG. 7).

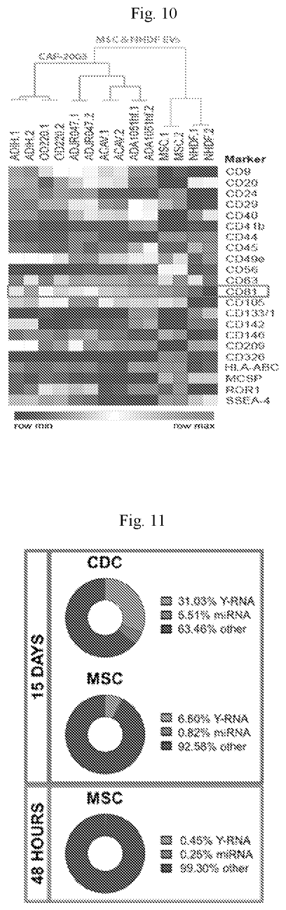

Protein immunoblot analysis confirmed the presence of EV surface markers CD81 and ?? in CDC-EVs (FIG. 8). Next, a more in-depth analysis of the expression and colocalization of surface markers CD81, CD63 and CD9 in CDC-EVs and MSC-EVs was performed using NanoView technology (FIG. 9). Finally, the presence of various surface markers on CDC-EVs and MSC-EVs was analyzed using the MACSPlex technology with the expression of typical exosome markers (CD63, CD81, CD9) as well as the CDC surface marker CD105 (FIG. 10). Interestingly, a higher expression of CD9 and CD81 and lower expression of HLA-class I (HLAABC) was observed in CDC-EVs compared to MSC-EVs.

### F. CDC-EVs and MSC-EVs have Distinct Y RNA and miRNA Profiles

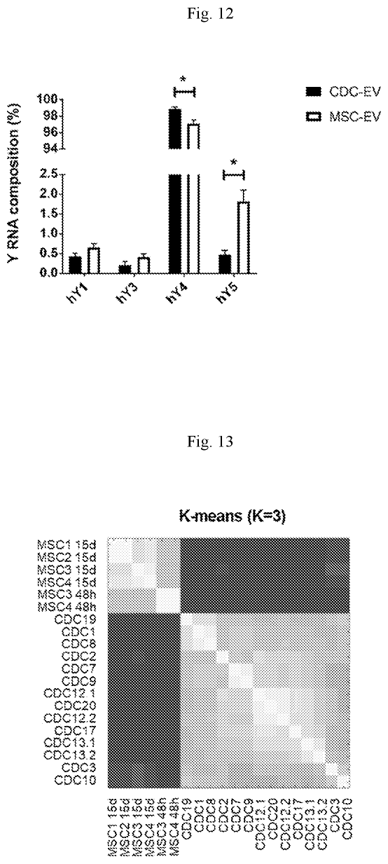

To identify biological differences between CDC-EVs derived from different donors and MSC-EVs, the present inventors performed small RNA sequencing. EVs from CDCs (n=5) cultured for 15 days in serum-free media showed the highest Y RNA and miRNA content compared to 15 days or 48 hours starved MSC-EVs (n=3) (FIG. 11). Human cells express 4 different non-coding Y RNA species: hY1, hY3, hY4 and hY5, with hY4 the most abundant in both CDC-EVs and MSC-EVs (FIG. 12). A significant relative increase of hY4 and decrease of hY5 were observed in CDC-EVs compared to MSC-EVs, respectively. However, the absolute amount of both hY4 and hY5 is the highest in CDC-EVs compared to MSC-EVs (FIG. 11). Interestingly, analysis of uniquely-alignable reads in CDC-EVs (n=12) showed that at least 50% were derived from miR-sized fragments of 20-23 bp long. In is contrast, in MSC-EVs (n=4) the 20-23 bp size range represented less than 25% of the uniquely-alignable reads (data not shown). Next, all uniquely-alignable reads of 20-23 bp in length were analyzed of which approximately 20-50% coincided with miR-22-3p. The remaining reads were used for further analysis. A repeated down-sampling technique was applied to equal the number of reads in each sample. For the comparison of CDC-EVs and MSC-EVs, 100-500 down-sampling trials to 20,000 reads per sample were completed followed by an unsupervised K-means clustering analysis for each trial. FIG. 13 shows that MSC-EVs clustered consistently separately from CDC-EVs. Interestingly, a minor subcluster of the 48 hour samples could be observed within the MSC-EV cluster.

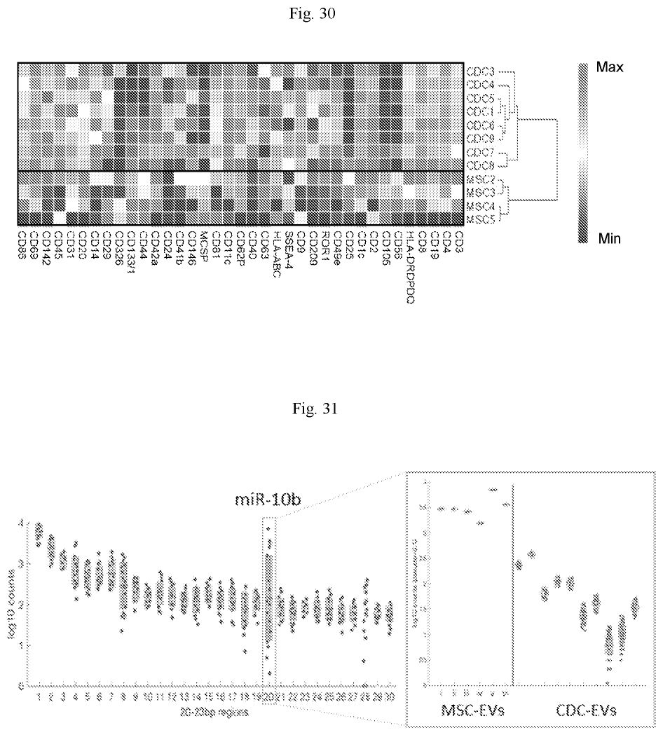

Referring to FIG. 30, to determine the compositional differences between CDC- and MSC-EVs, 15-day serum-free EV-enriched conditioned media was collected for protein (MACSPlex, Miltenyi) and RNA (small RNA-sequencing, Illumina) analyses. EV samples from both groups were probed for 37 different surface markers. Despite some variability between donors from the same group, EVs derived from CDCs and MSCs consistently clustered with their cell of origin. Specifically, CDC-EVs expressed higher levels of CD9, CD24, CD41b, and CD49e and decreased expression of CD326, CD133, CD44, CD105, and CD56 relative to MSC-EVs. To determine the contribution of miRNA to these profiles, the present inventors focused on reads of 20-23 bp in length. While most miRNA aligned consistently between groups, the present inventors observed one clear outlier: miR-10b (the 20th most abundant miR; FIG. 31); elevated miR-146a expression in CDC-EVs was confirmed (FIG. 43). Interestingly, the duration of conditioning positively correlated with miR-10b expression. MSCs collected from the same donor, but conditioned for 2 time periods, revealed lower miR-10b expression at 48-h (FIG. 31; MSC-EV iv and vi) compared to 15-days (FIG. 31; MSC-EV iii and v). Enriched expression of miR-10b in MSC-EVs was confirmed by qPCR (FIG. 32).

### G. miRNA Loading

mi RNAs are combined with different combinations and amounts of polycationic lipids and EVs, as well as in different orders of addition. RNA loading of EVs involves pre-mixing of miRNAs with polycationic lipids followed by addition of EVs.

EVs and mRNAs are combined with mRNA MAX transfection reagent and incubated to form a suspension of EV-miRNA-lipid hybrid, or an EV-liposome hybrid vesicle loaded with, or combined with, synthetic miRNAs, that combines (i) the protective and anti-inflammatory properties of EVs with (ii) the cell membrane-penetrating properties of lipofection reagent lipids.

### H. Evaluation and Determination of CDC Potency in an In Vivo MI Mouse Model

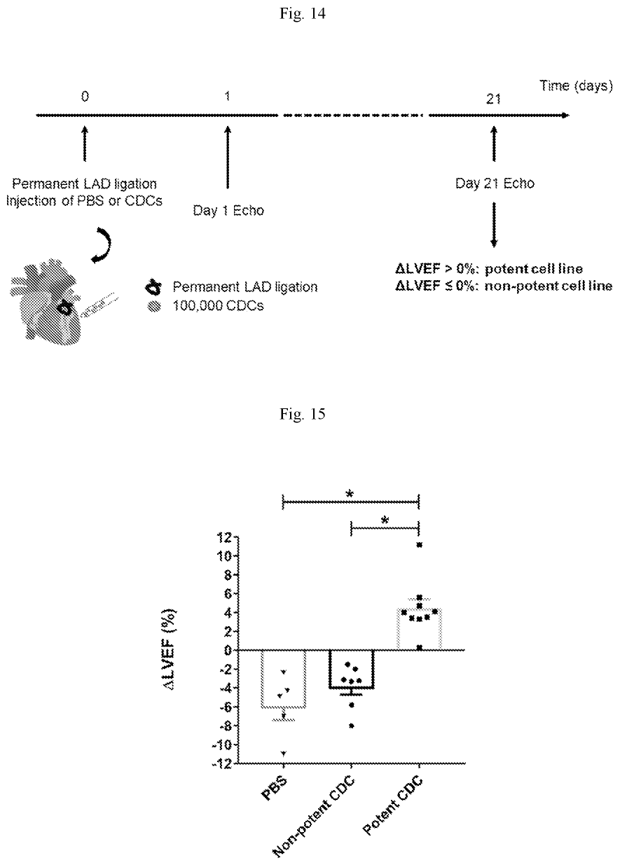

CDCs possess a range of regenerative potency after an ischemic cardiac event depending on the donor (18, 19). The present inventors determined potency by the post-myocardial infarction (MI) left ventricular ejection fraction (LVEF) after intramyocardial injection of CDCs compared to placebo (FIG. 14) as previously described (22). To calculate the LVEF, a long axis image of the LV in the B-mode was traced in the diastolic and systolic phase. From these traces, volumes and LVEF was calculated using the manufacturer's software. A significantly improved LVEF 21 days post-MI compared to PBS (n=5) was observed in mice injected with cells from 9 different CDCs donors (P<0.01), but not when mice were treated with CDCs from 7 non-potent donors (FIG. 15). Based on this assay, the present inventors identified CDCs as potent-CDCs when significant improvement in LVEF was observed after cell injection, or as non-potent CDCs when no significant improvement was observed. Mice treated with MSCs showed an improvement in LVEF similar to the mice treated with non-potent CDCs (data not shown).

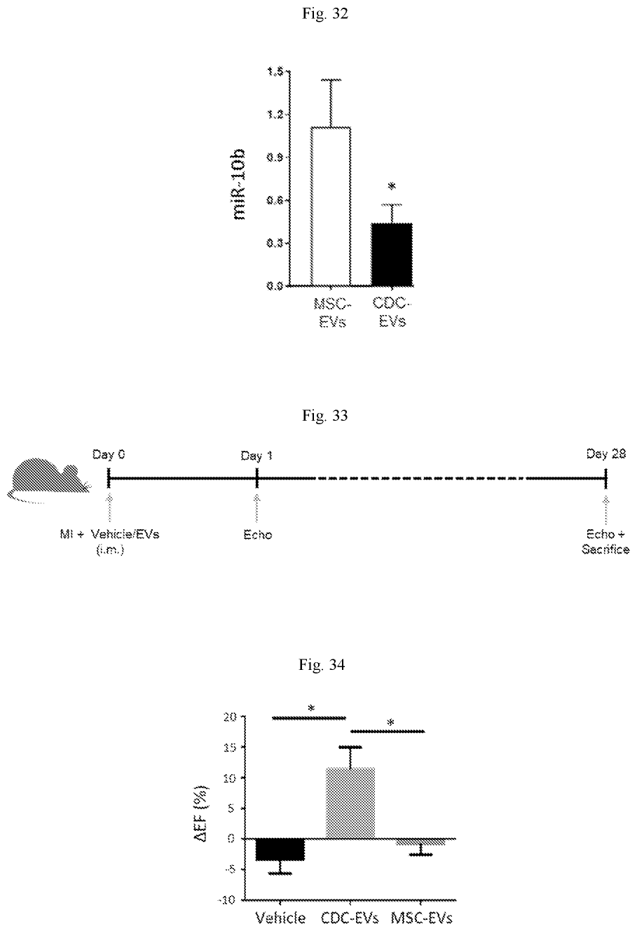

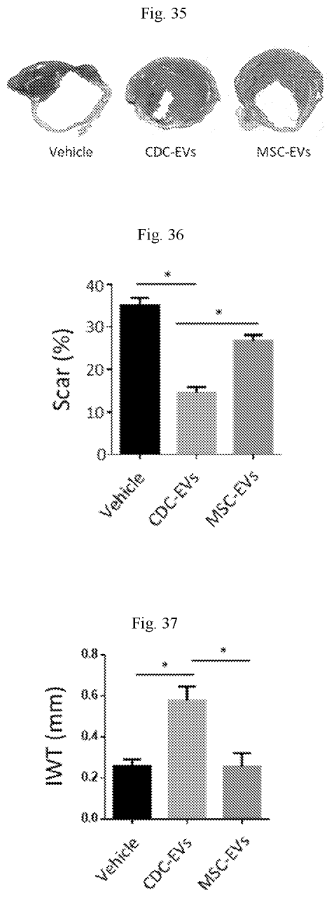

Referring to FIG. 33, the present inventors compared the efficacy of a single intramuscular injection of vehicle, CDC-EVs, or MSC-EVs in a mouse model of MI. In contrast to MSC-EVs, CDC-EVs improved cardiac function 4 weeks post-MI (FIGS. 34-35). These functional changes were associated with a reduction in scar size (FIG. 36) and an increase in infarct wall thickness (FIG. 37). Together, these data reveal the therapeutic superiority of CDC-EVs, relative to MSC-EVs, when given immediately post-MI.

### I. Potent-Derived CDC-EVs Polarize Macrophages Toward an Anti-Inflammatory Phenotype

It was previously shown that CDC-EVs increase phagocytic capabilities of macrophage and reduce the expression of pro-inflammatory genes, polarizing the macrophages to a phenotype that improves cardiac healing after an ischemic event. To evaluate the correlation between the potency of different CDCs (MI mouse model) and the ability of their EVs to polarize macrophages to a pro-regenerative phenotype, the present inventors tested EVs on activated peritoneal macrophages.

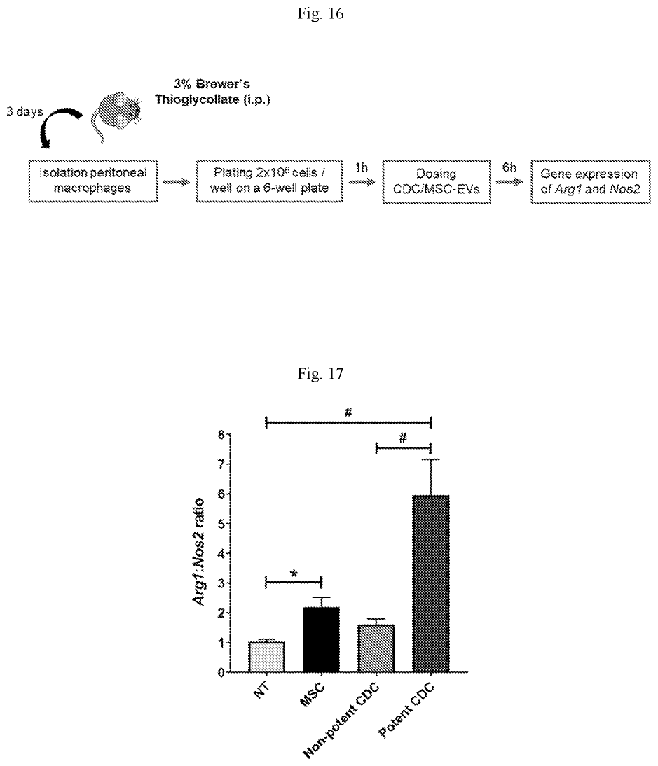

C57BL/6 female mice were treated with 3% Brewer's thioglycollate to induce an influx of partially activated macrophages into the mice peritoneum followed by a peritoneal lavage after 3 days to collect macrophages and seed them on culture plates (FIG. 16). After settling, peritoneal macrophages were left untreated or treated with MSC-EVs (n=3) or with potent- (n=5) or non-potent-derived (n=5) CDC-EVs. After 6 hours incubation, macrophages were harvested to evaluate the gene expression of the pro-inflammatory M1 marker Nos2 and the anti-inflammatory M2 marker Arg1. The Arg1:Nos2 expression ratio of non-treated macrophages was normalized to 1 and used as reference (FIG. 17).

After treatment of peritoneal macrophages with EVs derived from potent CDCs, a statistically significant increase of the Arg1:Nos2 ratio (P<0.01) was observed when compared with non-potent CDC-EVs, suggesting that potent CDC-derived EVs are capable of modulating the polarity of macrophages towards the M2 (anti-inflammatory) phenotype (FIG. 17). A comparable Arg1:Nos2 ratio was detected between EVs derived from MSCs and non-potent derived CDCs. However, treatment of macrophages with MSC-EVs induced a smaller but significant (P<0.05) increase of the Arg1:Nos2 ratio than potent CDC-EVs compared to nontreated macrophages (FIG. 17).

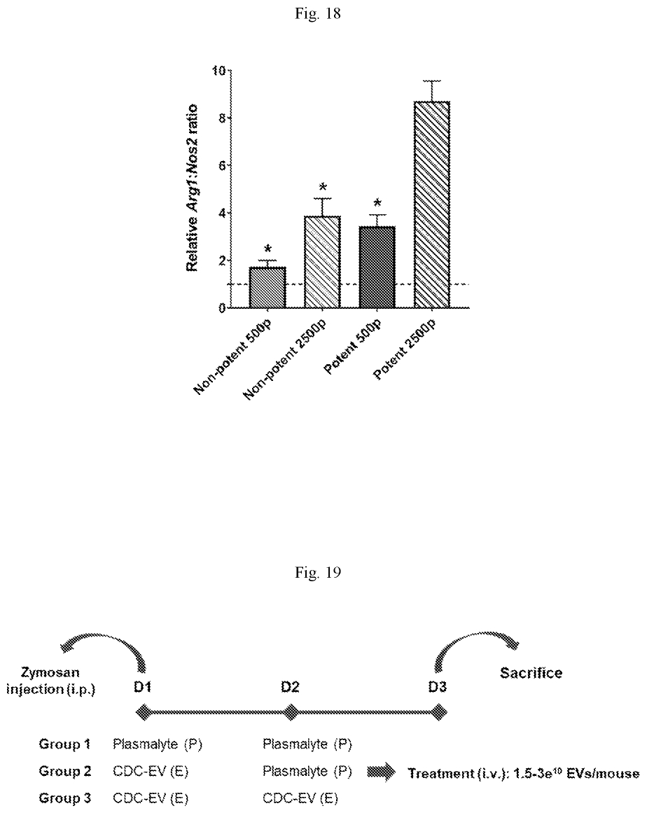

Macrophage immunomodulatory capabilities of CDC-EVs were also confirmed using rat macrophages. Wistar-Kyoto female rats 2-6 months old were treated as above and peritoneal macrophages collected 3 days after Brewer's thioglycollate injection. Peritoneal macrophages were seeded in culture plates and left untreated or treated with 500 or 2500 particles/cell of potent- (n=4) or non-potent-derived (n=3) CDC-EVs (FIG. 18). A dose dependent increase in the expression of Arg1 without significant changes in Nos2 expression compared to non-treated macrophages was observed after macrophage treatment with CDC-EVs. The increased Arg1:Nos2 ratio was significantly higher when macrophages were treated with 2,500 particles/cell of potent CDC-EVs compared to treatment with 500 particles/cell of potent-derived CDC-EVs and 500 or 2,500 particles/cell of non-potent-derived CDC-EVs. Interestingly, macrophages treated with 500 particles/cell of potent CDC-EVs showed a similar Arg1:Nos2 ratio as macrophages treated with 2500 particles/cell of non-potent CDC-EVs.

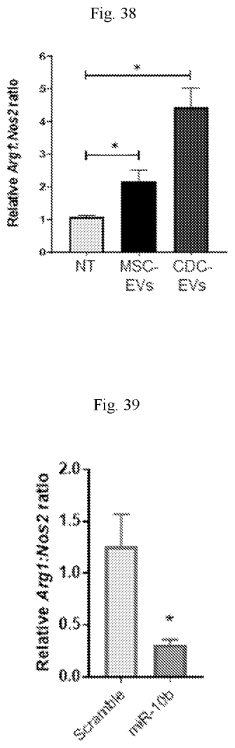

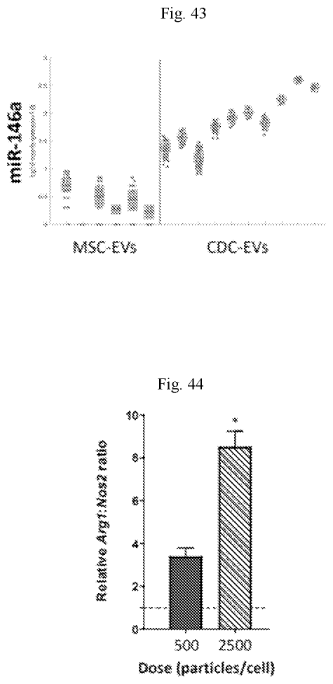

Referring to FIG. 44, peritoneal Mϕ, which were isolated from thioglycolate-stimulated mice, were plated and treated with varying doses of CDC-EVs. Six hours later, RNA was isolated and the relative expression of Arg1 and Nos2 gene expression were analyzed. The present inventors observed a dose-dependent increase in Arg1/Nos2 ratio when increasing EV dose from 500 particles/cell to 2500 particles/cell. Based on these results, the present inventors compared the efficacy of MSC-EVs and CDC-EVs (standardized dose of 2500 particles/cell) to modify the Arg1/Nos2 gene expression profile in Mϕ. While both EVs elicited upregulation of the Arg1/Nos2 ratio, the present inventors found that CDC-EVs were more potent (FIG. 38). Addition of miR-10b mimic confirmed the inhibitory effect of this miR by reducing the Arg1/Nos2 ratio relative to miR scramble control (FIG. 39).

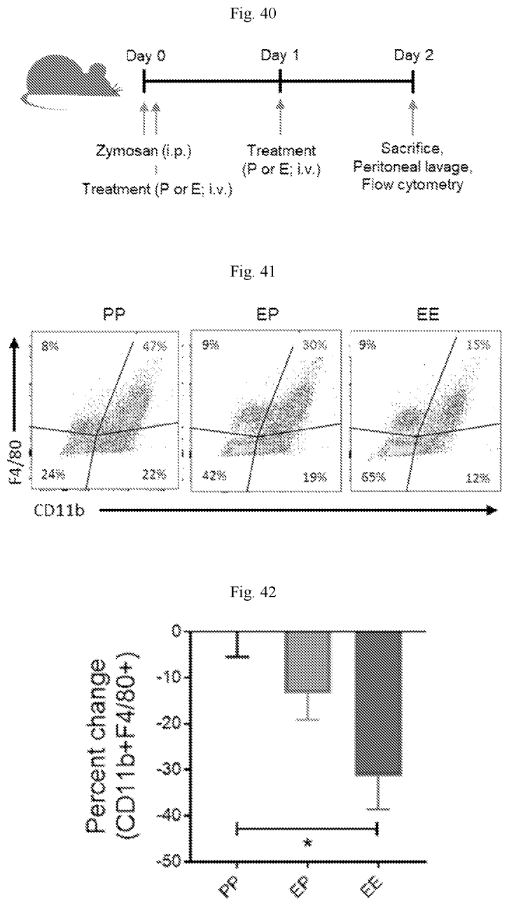

The present inventors continued to test the efficacy of the more potent EV population (CDC-EVs) in a mouse model of acute peritonitis. To do so, mice were stimulated with Zymosan (i.p.), treated with placebo (P) or CDC-EVs (E) on days 0 and 1 (PP: placebo days 0 and 1; PE: placebo day 0, CDC-EVs day 1; EE: CDC-EVs days 0 and 1), and then sacrificed on day 2 (FIG. 40). Peritoneal cavities were flushed, and inflammatory cells profiled by flow cytometry. Interestingly, the present inventors observed a marked decrease in peritoneal Mϕ (CD11b+F4/80+) in mice that received 2 sequential doses of CDC-EVs (EE), relative to a single dose (EP) or placebo only (PP) (FIGS. 41-42). Together, these data demonstrate that EVs alter the gene expression profile of inflammatory M and that CDC-EVs have the capacity to suppress peritoneal M influx in a model of acute peritonitis.

### J. CDC-EVs Reduce Macrophage Recruitment In Vivo in a Sepsis Mouse Model

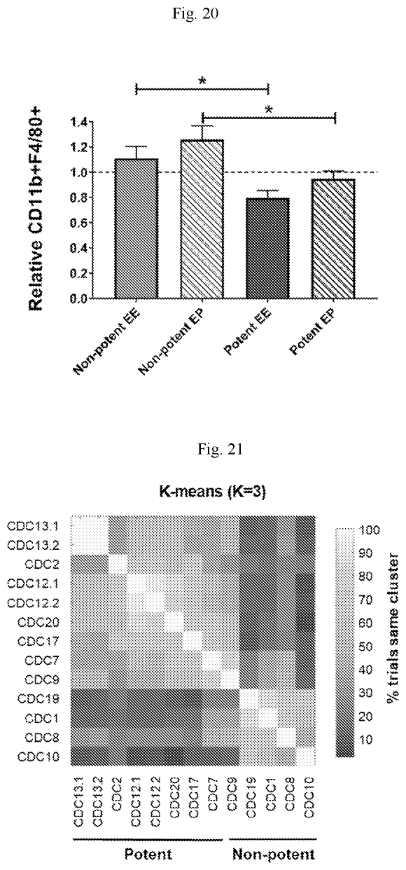

Since it was observed that EVs derived from potent CDCs polarize macrophages from a pro-inflammatory M1-like to a pro-regenerative M2-like phenotype in vitro, the present inventors examined if CDC-EVs would modulate the inflammatory response in vivo. To test this hypothesis, the present inventors induced an acute inflammatory insult in C57BL/6 mice through i.p. injection of 1 mL of Zymosan A solution (100 μg/mL) on day 1. This well-known and self-resolving model of sepsis is used to study inflammation and mediators that induce downregulation of the inflammatory response (23). Mice were i.v. treated immediately after Zymosan A injection with placebo (P) or with 1.5-3×10¹⁰ CDC-EVs (E) on day 1 and day 2 (PP and EE groups) or with 1.5-3×10¹⁰ CDC-EVs on day 1 and placebo on day 2 (EP group) (FIG. 19). At day 3, mice were sacrificed and a peritoneal lavage was performed to quantify the recruitment of activated macrophages as response to the acute inflammation. Mice treated with 2 doses of potent-derived CDC-EVs (EE) had a significant reduction in influx of activated CD11b⁺F4/80⁺ macrophages compared to mice treated with non-potent-derived CDC-EVs (EE) (FIG. 20). In addition, a 20% reduction of CD11b⁺F4/80⁺ peritoneal macrophages was observed in the potent EE-treated group compared to mice treated with placebo (PP). However, this reduction was not significant. Interestingly, mice treated with a single dose of potent-derived CDC-EVs (EP) barely experienced a reduction in is influx of CD11b⁺F4/80⁺ macrophages, suggesting that only repeated dosing was effective to reduce the inflammatory response.

### K. An Increased miR-345-5p Expression in Potent-Derived CDC-EVs Compared to Non-Potent-Derived CDC-EVs and MSC-EVs

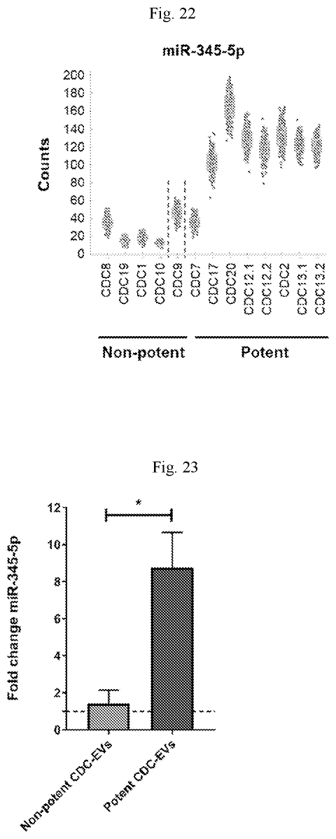

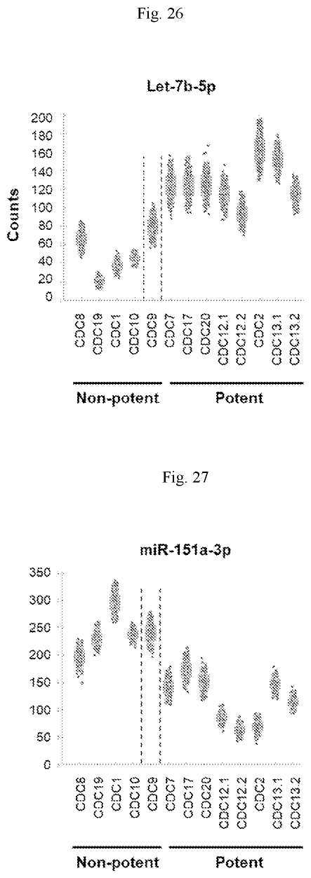

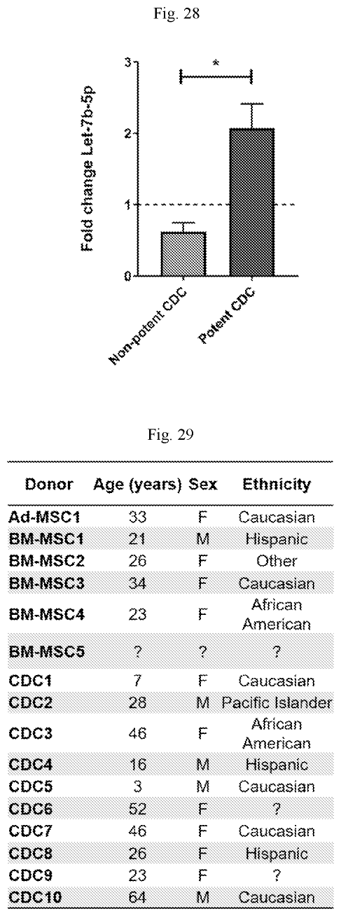

The different ability of EVs obtained from potent and non-potent CDCs to modulate macrophage responses suggests for the presence of different cargos in each kind of EVs. Unsupervised blinded clustering analysis on CDCs small RNA sequencing data was performed and miRNAs were identified to be differentially expressed between potent- and non-potent-derived CDC-EVs (FIG. 21). FIG. 21 shows the unsupervised K-means clustering of only CDC-EV samples with 500 downsampling trials to 60,000 reads per sample. Four of the 5 non-potent CDC-EVs, clustered separately from the remaining 6 potent CDC-EV samples showing that potent and non-potent derived CDC-EVs contain a distinct miRNA cargo. More specifically, miR-345-5p was significantly higher in potent-derived CDC-EVs compared to non-potent-derived CDC-EVs (FIG. 22). The increased expression was confirmed with qPCR and showed an 8.8-fold increase in potent-derived CDC-EVs compared to non-potent-derived CDC-EVs and MSC-EVs (FIG. 23). In addition, two other miRNAs were differentially expressed between potent and non-potent-derived CDC-EVs: miR-151a-3p and let-7b-5p. An increased let-7b-5p and decreased miR-151a-3p expression were observed in potent-derived CDC-EVs compared to non-potent-derived CDC-EVs (FIG. 26 and FIG. 27). However, the increased let-7b-5p expression in potent CDC-EVs was confirmed with qPCR but not the decreased miR-151a-3p expression levels (FIG. 28).

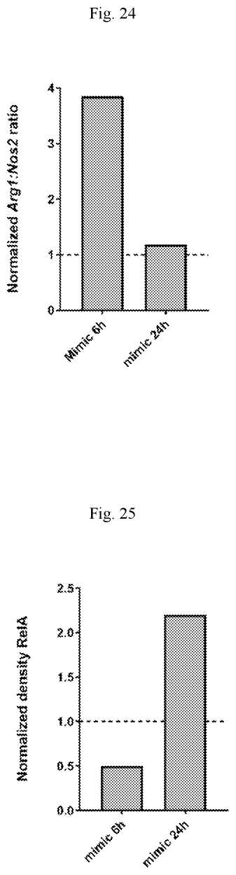

It has been previously shown that miR-345 is differentially expressed between proinflammatory CD16⁺ and CD16⁻ of monocytes and that it regulates the expression of RelA and indirectly the expression of important inflammatory mediators downstream of RelA (24). The present inventors hypothesized that the increased expression of miR-345-5p in potent-derived CDC-EVs supports the enhanced immunomodulatory capacity and thus the increased Arg1:Nos2 expression in treated macrophages. To investigate this hypothesis, the present inventors transfected rat macrophages with miR-345-5p mimic or miR-scramble and evaluated the Arg1 and Nos2 expression after 6 and 24 hours. An increase in the Arg1:Nos2 ratio is seen in macrophages transfected with miR-345-5p mimic although this increase was lower than the one observed in macrophages treated with potent-derived CDC-EVs (FIG. 18 and FIG. 24), suggesting that other molecules present in CDC-EVs might also contribute during macrophage polarization. The Arg1:Nos2 ratio increase observed after miR-345-5p mimic transfection was transient and diminished after 24 hours.

RelA protein levels were analyzed by Western blot in macrophages 6 or 24 hours after transfection with miR-345-5p mimic or miR-scramble (FIG. 25). In accordance with the Arg1:Nos2 ratio observed, the RelA protein levels were decreased after 6 hours but were restored 24 hours after the treatment.

These data suggest that miR-345-5p present in CDC-EVs contribute to the polarization of macrophages from a pro-inflammatory phenotype to a more pro-regenerative profile which could contribute to the anti-inflammatory effects observed in vivo after the delivery of CDC-EVs.

### L. Study Approval

Human heart tissues were obtained under an Institutional Review Board (IRB)-approved protocol (Cedars-Sinai Medical Hospital, Los Angeles, Calif.).

All mouse and rat experiments were approved by the Institutional Animal Care and Use Committee (IACUC) of Cedars-Sinai Medical Hospital prior to commencement and related procedures were conducted according to IACUC regulations.

### M. In Vivo MI Mouse Model

Male SCID mice (Balb/c mice, Janvier Labs) were anesthetized with ketamine-xylazine (80-120 mg/kg, 10-16 mg/kg body weight respectively, intraperitoneally (IP)), intubated and ventilated at 150 breaths per minute (0.25 mL tidal volume, MiniVent, Harvard Apparatus). Body temperature was monitored and maintained at 37° C. using a rectal probe and heating pad (TC-1000, CWE Inc.). The LAD was permanently ligated (7-0 silk suture) to induce an MI as previously described (22). Immediately after ligation and once cardiac tissue became pale, 100,000 CDCs or PBS were injected into the infarct border zone. In total, 16 mice were injected with cells from the same donor. After LAD ligation and cell injection, all layers of muscle and skin were closed using a 6.0 ticron suture, the wound was treated with an antiseptic, and an analgesic (buprenorphine, Schering-Plough, 0.1 mg/kg subcutaneously) was administered during the first two days.

Echocardiographic measurements were performed at day 1 and day 21 post LAD ligation. Mice were sedated with 1.5% isoflurane (Ecuphar) and standard views were obtained in B-mode using a 30 MHz probe on a Vevo 2100 scanner (VisualSonics Vevo). Image analysis was performed using the manufacturer's software and LVEF was calculated using the acquired B-mode images.

### N. miRNA Sequencing and Data Analysis

Sequencing was performed by the Cedars-Sinai Genomics Core (Los Angeles, Calif.). Total RNA of CDC-EVs (n=12) and MSC-EVs (n=4) was extracted using the miRNeasy Serum/Plasma kit (Qiagen). Library construction was performed according to the manufacturer's protocol using the TruSeq small RNA Library Kit (Illumina). Briefly, 1 μg total RNA was used as starting material and adapters were ligated to the 3′ and 5′ ends of the small RNAs, sequentially followed by reverse transcription for conversion into cDNA. The resulting cDNA was enriched (PCR) and gel purification was performed prior to pooling of indexed library cDNAs and assessment for quality using the Agilent Bioanalyzer 2100. RNA-seq libraries were sequenced on a NextSeq 500 (Illumina, 75 bp read length, average sequencing depth of 10M reads/sample). The raw, demultiplexed sequencing signal (FASTQ) was pre-processed accordingly. Briefly, adaptors and low-quality bases were trimmed, reads of <16 nucleotides were excluded from further analysis. Next, the filtered reads were aligned to the miRbase (Release v2.1) mature and hairpin databases sequentially using Bowtie v1.2 toolkit (42) and quantified with mirDeep2 software (v2.0.0.8) (43). The counts of each miRNA molecule were normalized based on the total read counts for each sample.

Reads of 20-23 bp were aligned using the BWA software (v.0.7.12) (44). All uniquely alignable reads were extracted followed by repeated downsampling (100-500 downsampling trials per sample) to normalize the number of uniquely-alignable reads per CDC-EV and MSCEV sample. The Samtools program was used to randomly sample reads from each CDC-EV and MSC-EV entry. The independent K-means and hierarchical agglomerative clustering techniques with a cluster number of 3 were used to analyze CDC-EV and MSC-EV samples. To compare MSC-EV with CDC-EV samples, 100-500 downsampling trials to 20,000 uniquely-alignable reads were performed. To compare potent with non-potent CDC-EVs, 500 downsampling trials to 60,000 uniquely-alignable reads were performed.

### O. Surface Marker Profile Using MACSPlex Technology

Approximately 1e10 CDC-EVs were profiled for surface markers using the MACSPlex Exosome Kit (Miltenyi) that allows detection of 37 surface epitopes that are known to be present on various EVs plus two isotype controls. The kit comprises of antibody-coated MACSPlex Exosome Capture Beads which are fluorescently labeled (FITC and PE) by specific binding of the MACSPlex Exosome Detection Reagent. The detection reagent was combined to create a cocktail comprising of detection reagent for CD9, CD63, and CD81 (APC) for broad exosome staining. The protocol was performed according to manufacturer's recommendations. Flow cytometry was performed on a MACSQuant Analyzer 10 (Miltenyi). Flow cytometric acquisition and data analysis were done using the Express Mode setting. First, the samples were automatically measured, and the different bead populations were automatically gated for each capture bead which was linked to a specific epitope. Secondly, APC fluorescence values for markers CD9, CD63 and CD81 were collected for each capture bead population. The APC values for markers that had fluorescence detection above the isotype control beads were retained and normalized to the mean of the fluorescence of the exosome marker beads CD9, CD63 and CD81. Heat map and hierarchical cluster analyses were performed using the MORPHEUS versatile matrix visualization and analysis software online (https://software.broadinstitute.org/morpheus/). The one minus Pearson's correlation setting was used to generate the clustering.

### P. In Vitro Peritoneal Macrophage Assay

C57BL/6 mice and Wistar-Kyoto rats were intraperitoneal injected with Brewer's thioglycollate solution (3% in PBS) to induce a transient influx of inflammatory cells. After 3 days, the peritoneal macrophages were isolated through a peritoneal lavage with 0.75% EDTA (w/v in PBS). Cells were filtered through a 70-μm mesh filter followed by treatment with ACK lysis buffer (Thermo Fisher Scientific) for 1 minute at room temperature to lyse the red blood cells. Cells were resuspended in macrophage medium (RPMI 1640 with 10% FBS and 1% Pen/Strep) and 2×10⁶ cells were plated per well on a 6-well plate. Following a 1-hour incubation at 37° C., 5% CO₂, macrophages were dosed with EVs (500, 1000, 2500 or 5000 EVs per cell) and incubated for 6 hours at 37° C., 5% CO₂. Finally, macrophages were washed with PBS, cell lysates were recovered in RLT lysis buffer (QIAGEN) and stored at −80° C. before performing total RNA isolation.

### O. In Vivo Peritoneal Macrophage Assay

C57BL/6 mice were intraperitoneally injected with 1 mL Zymosan A solution (100 μg/ml in PBS) to induce a transient influx of inflammatory cells. The mice were treated with 1.5-3e10 EVs in 200 μl plasmalyte (E) or with 200 μl plasmalyte (P) by tail vein injection. On day 2, the mice were treated with 1.5-3e10 EVs in 200 μl plasmalyte (E) or with 200 μl plasmalyte (P) by tail vein injection. On day 3, peritoneal macrophages were isolated through a peritoneal lavage with 0.075% EDTA (w/v in PBS). The cells were filtered through a 70-μm mesh filter followed by treatment with ACK lysis buffer (Thermo Fisher Scientific) for 1 minute at room temperature to lyse the red blood cells. The cells were washed with FACS buffer (PBS with 0.075% EDTA 1% Equafetal serum) and centrifuged. The pellet was dissolved in FACS buffer with fluorochrome-conjugated antibodies and incubated for 30 minutes at 4° C. in the dark. Samples were analyzed on a LSRII flow cytometer (BD Biosciences) with at least 10,000 recorded events. Single stains and unstained samples were used as controls. Data were analyzed with FlowJo 10 software (FlowJo LLC). A total of 6 mice per group per CDC-EV donor and a total of 24 mice in the control group (PP) were analyzed.

### R. Transfection of miRNA Mimics in Peritoneal Macrophages

Peritoneal macrophages were collected and 2e6 macrophages were seeded as described previously. After 1 hour incubation of peritoneal macrophages, microRNA mimics (miR-345-5p mimic and miR-scramble, mirVana) were directly transfected into peritoneal macrophages utilizing DharmaFECT 4 (GE/Dharmacon), according to the manufacturer's protocol. Briefly, miRNA was mixed with DharmaFECT 4 solution in serum-free basal RPMI media and incubated for 20 minutes at room temperature while shaking. The appropriate volume of complete media was added to reach a final miRNA concentration of 25 nM. The miRNA was added to the cells for 6 hours or 24 hours, followed by RNA harvest (Arg1, Nos2 evaluation) and protein harvest (RelA evaluation).

### S. Quantitative Real-Time PCR (qPCR)

Total RNA was isolated using the RNeasy Mini Plus Kit (QIAGEN) followed by reverse transcription using the High-Capacity RNA-to-cDNA kit (Thermo Fisher Scientific) according to manufacturer's protocol. Real-time qRT-PCR was performed using TaqMan Fast Universal PCR Mastermix and TaqMan Gene Expression Assays for Hprt, Nos2 and Arg1 on a QuantStudio 12K Flex Real-Time PCR System (Thermo Fisher Scientific). All reactions were run in triplicate and results were expressed as 2^{-ΔΔCt}. Relative gene expression was normalized to the housekeeping gene Hprt and normalized gene expressions of EV-treated groups were compared to the non-EV treated (NT) group.

Referring to FIG. 32, TaqMan Fast Universal PCR Mastermix and TaqMan miRNA Assays primers were used to detect miR-23a-3p and miR-10b-5p (QuantStudio 12 K Flex, Thermo Fisher Scientific). All reactions were run in triplicate and results were expressed as 2^{-ΔΔCt}. Relative gene expression was normalized to miR-23a-3p.

### T. Western Blot Analysis

Macrophages were washed with cold PBS and RIPA containing HALT protease and phosphatase inhibitor cocktail (ThermoScientific) was added. The cells were scraped, lysates were collected, incubated on ice for 30-60 minutes, followed by centrifugation for 15 minutes at 10,000 g at 4° C. The protein supernatants were collected and stored at −80° C. Protein concentrations were measured using a BCA assay (ThermoScientific). Protein samples were prepared for gel electrophoresis (Mini-PROTEAN TGX Precast gel, 4-15%, Bio-Rad) according to the manufacturer's protocol. A normalized final loading concentration between 5 and 10 μg per well was used. Proteins were then transferred to a nitrocellulose membrane (Bio-Rad) for immunoblotting with designated antibodies (Actin, 1:2,000, Sigma-Aldrich; RelA, 1:1,000, Cell signaling technologies). Finally, the specific antigens were visualized using enhanced chemiluminescence (ECL) detection reagents (Thermo Scientific) and imaged (ChemiDoc MP imaging system, Bio-Rad). Semi-quantitative analysis of the imaged bands was performed using densitometric analysis in ImageJ. Protein levels of actin, an abundant cytoskeletal protein, were used as a loading control.

### U. Statistical Analysis

All data are presented as mean f standard error of the mean (SEM). Column statistics were applied to all data including a Shapiro-Wilk normality test. For normally distributed data, intergroup differences were analyzed using a two-tailed unpaired t-test or a one-way ANOVA followed by a Bonferroni post-hoc test. Non-parametric tests (Mann-Whitney test or Kruskal-Wallis test followed by a Dunn's post-hoc test) were used for non-normally distributed data. All analyses were performed using Prism 7 software (GraphPad Software) and only differences with a P<0.05 were considered statistically significant.

### V. Discussion

Cardiosphere-derived cells (CDCs) and their secreted extracellular vesicles (CDC-EVs) have been shown to induce cardiac repair in the acute and chronic phase after MI. In addition, it has been previously shown that CDC-EVs exert their effect by modulating macrophages into a reparative, cytoprotective phenotype distinct from M1 and M2 macrophages. Increased levels of miR-181b in CDC-EVs compared to EVs derived from is fibroblasts was identified to play a key role in polarizing the macrophages away from a pro-inflammatory M1 phenotype (13). Here, the present inventors demonstrate that CDCEVs derived from potent cell lines polarize macrophages towards an anti-inflammatory phenotype, which may confer some of the reported beneficial immune effects in vivo during cardiac repair. Interestingly, CDC-EVs from non-potent cell lines and MSC-EVs are less capable of this effect in an in vitro macrophage assay. The difference in immunomodulatory bioactivity between potent and non-potent derived CDC-EVs is confirmed by an in vivo sepsis model with a reduction of peritoneal macrophage recruitment in animals treated with EVs from potent CDCs. Importantly, small RNA sequencing analysis revealed a distinct EV content profile with a higher absolute Y-RNA content in CDC-EVs, regardless of CDC potency, and a marked difference in miRNA composition compared to MSC-EVs. Further analysis of the miRNA content identified a signature of 3 miRNAs that are differentially expressed in EVs derived from potent and non-potent CDCs.

Y RNA fragments are small non-coding RNA fragments of which four molecules are described in humans (Y1, Y3, Y4 and Y5). These Y RNAs were initially discovered to form a complex with the ribonucleoproteins Ro60 and La proteins (25, 26). These proteins are important targets for autoimmune responses in rheumatic diseases such as systemic lupus erythematosus and Sjögren's Syndrome (27, 28). All four Y RNA molecules have a similar stem-loop secondary structure. Y RNA function has been implicated in DNA replication and regulation of the degradation of misfolded RNAs. Recently, it has been found that Y RNAs are abundantly present in the extracellular environment. Moreover, an enrichment is found in EVs relative to intracellular levels (29-31). Interestingly, most of the functions of Y RNA present in EVs is linked to both pro- and anti-inflammatory effects (7, 32, 33). Haderk et al. showed that chronic lymphocytic leukemia (CLL) derived EVs in the plasma contains increased levels of hY4 RNA species. Treatment of monocytes with CLL-EVs or hY4 alone resulted in release of CCL-2, CCL-4, IL-6 and expression of PD-L1 which could explain tumor-related inflammation and immune escape through PD-L1 signaling (32). In addition, is has been previously shown that CDC-EVs contain a higher hY4 content. Transfer of these CDC-EVs and thus hY4 to macrophages leads to an upregulation of IL-10 and TNF-α. This translates to cytoprotection and reduced infarct size in an in vivo MI animal model (7). Here, the present inventors showed a higher Y RNA content in 15 days CDC-EVs, irrespective from potency, compared to 15 days and 48 hours MSC-EVs, in particular an increased hY4 which confirms earlier observations. This could partially explain the increased is immunomodulatory effect of CDC-EVs compared to MSC-EVs in the in vitro macrophage assay.

Next, the present inventors showed an increased immunomodulatory capacity of EVs derived from potent CDCs compared to EVs derived from non-potent CDCs and MSC-EVs in an in vitro macrophage assay. The correlation of potency of CDC donors between the in vivo MI mouse model and the in vitro macrophage assay suggests that the cytoprotection and improved EF seen in the MI model can partially be explained by the immunomodulatory effect of potent CDC-EVs. This confirms earlier reports that CDCs and CDC-EVs improve the cardiac function after an ischemic insult both in in vivo animal models (3, 34-38) as well as in clinical trials (CADUCEUS) (20). In addition, multiple reports describe the immunomodulatory capacity of CDCs and CDC-EVs and especially their potential to polarize macrophages into an anti-inflammatory phenotype that promotes repair (1, 13, 39, 40). The present inventors confirmed the in vitro observation in an in vivo sepsis model with a reduced recruitment of CD11b⁺F4/80⁺ macrophages after 2 systemic treatments with potent CDC-EVs compared to non-potent CDC-EVs.

Small RNA sequencing revealed an increased presence of miR-345 in EVs derived from potent CDCs compared to non-potent CDC-EVs which was confirmed with qPCR. When macrophages were transfected with a miR-345 mimic, a comparable effect is seen when macrophages are treated with potent-derived CDC-EVs in the in vitro macrophage assay. One of the targets of miR-345 is the transcription factor RelA, also known as p65, which is part of the NF-κB family. NF-κB signaling is involved in many different cellular processes and acts as a major mediator in inflammatory responses. The NF-κB family members are all transcription factors that regulate the expression of pro-inflammatory genes in innate immune cells, the function of inflammatory T cells and the activation of inflammasomes. Deregulation of NF-κB signaling leads to chronic inflammatory diseases such as rheumatoid arthritis, inflammatory bowel disease, multiple sclerosis, systemic lupus erythematosus, atherosclerosis, etc. (41). Dang et al. made the correlation between increased miR-345 expression in CD16⁻ monocytes and a decrease in RelA protein levels (24).

Important targets of the transcription factor RelA are CCL5, GCH1, LYN, SYTL1 and TNF-α which are all implicated in the inflammatory response. The authors concluded that the pro-inflammatory phenotype of the CD16⁺ monocytes may be partly mediated by a reduction in the expression of miR-345 and an increase of RelA protein levels (24). This observation suggests that the polarization of peritoneal macrophages towards an antiinflammatory is phenotype after treatment with potent-derived CDC-EVs is partially due to the increased miR-345 expression and subsequent decrease of RelA.

In addition, the present inventors identify miR-10b, which discriminates the miRNA population between MSC-EVs (enriched) and CDC-EVs.

In conclusion, the potency of CDC donors and their EVs plays an important role in their ability to polarize macrophages towards an anti-inflammatory, healing phenotype. Moreover, it was shown that potent-derived CDC-EVs have a superior immunomodulatory effect compared to MSC-EVs. This study identifies cargos inside CDC-EVs critical for their immunomodulatory activities. In addition, the identification of RNAs, Y-RNA and miRNAs, relevant for controlling pro-inflammatory responses paves the way for engineering cells and EVs and obtain products with a consistent high potency. The EVs engineered to be loaded with, inter alia, miR-345, according to one embodiment of the present invention is based on this discovery and addresses the need in the art for novel EVs having a consistent high potency.

### W. References

1. de Couto G, Liu W, Tseliou E, Sun B, Makkar N, Kanazawa H, et al. Macrophages mediate cardioprotective cellular postconditioning in acute myocardial infarction. J Clin Invest. 2015; 125(8):3147-62.
2. Kreke M, Smith R R, Marban L, Marban E. Cardiospheres and cardiosphere-derived cells as therapeutic agents following myocardial infarction. Expert Rev Cardiovasc Ther. 2012; 10(9):1185-94.
3. Tseliou E, de Couto G, Terrovitis J, Sun B, Weixin L, Marban L, et al. Angiogenesis, cardiomyocyte proliferation and anti-fibrotic effects underlie structural preservation post-infarction by intramyocardially-injected cardiospheres. PLoS One. 2014; 9(2):e88590.
4. Ibrahim A G, Cheng K, Marban E. Exosomes as critical agents of cardiac regeneration triggered by cell therapy. Stem Cell Reports. 2014; 2(5):606-19.
5. Terrovitis J V, Smith R R, Marban E. Assessment and optimization of cell engrafiment after transplantation into the heart. Circ Res. 2010; 106(3):479-94.
6. Barile L, Lionetti V, Cervio E, Matteucci M, Gherghiceanu M, Popescu L M, et al. Extracellular vesicles from human cardiac progenitor cells inhibit cardiomyocyte apoptosis and improve cardiac function after myocardial infarction. Cardiovasc Res. 2014; 103(4):530-41.
7. Cambier L, de Couto G, Ibrahim A, Echavez A K, Valle J, Liu W, et al. Y RNA fragment in extracellular vesicles confers cardioprotection via modulation of IL-10 expression and secretion. EMBO Mol Med. 2017; 9(3):337-52.
8. Thery C, Zitvogel L, Amigorena S. Exosomes: composition, biogenesis and function. Nat Rev Immunol. 2002; 2(8):569-79.
9. Davies L C, Rosas M, Smith P J, Fraser D J, Jones S A, Taylor P R. A quantifiable proliferative burst of tissue macrophages restores homeostatic macrophage populations after acute inflammation. Eur J Immunol. 2011; 41(8):2155-64.
10. Frangogiannis N G, Smith C W, Entman M L. The inflammatory response in myocardial infarction. Cardiovasc Res. 2002; 53(1):31-47.
11. Mills C D, Kincaid K, Alt J M, Heilman M J, Hill A M. M-1/M-2 macrophages and the Th1/Th2 paradigm. J Immunol. 2000; 164(12):6166-73.
12. Troidl C, Mollmann H, Nef H, Masseli F, Voss S, Szardien S, et al. Classically and alternatively activated macrophages contribute to tissue remodelling after myocardial infarction. J Cell Mol Med. 2009; 13(9B):3485-96.
13. de Couto G, Gallet R, Cambier L, Jaghatspanyan E, Makkar N, Dawkins J F, et al. Exosomal MicroRNA Transfer Into Macrophages Mediates Cellular Postconditioning. Circulation. 2017; 136(2):200-14.
14. Lai P, Chen X, Guo L, Wang Y, Liu X, Liu Y, et al. A potent immunomodulatory role of exosomes derived from mesenchymal stromal cells in preventing cGVHD. J Hematol Oncol. 2018; 11(1):135.
15. Pacienza N, Lee R H, Bae E H, Kim D K, Liu Q, Prockop D J, et al. In Vitro Macrophage Assay Predicts the In Vivo Anti-inflammatory Potential of Exosomes from Human Mesenchymal Stromal Cells. Mol Ther Methods Clin Dev. 2019; 13:67-76.
16. Reis M, Mavin E, Nicholson L, Green K, Dickinson A M, Wang X N. Mesenchymal Stromal Cell-Derived Extracellular Vesicles Attenuate Dendritic Cell Maturation and Function. Front Immunol. 2018; 9:2538.
17. Wang M, Yuan Q, Xie L. Mesenchymal Stem Cell-Based Immunomodulation: Properties and Clinical Application. Stem Cells Int. 2018; 2018:3057624.
18. Cheng K, Malliaras K, Smith R R, Shen D, Sun B, Blusztajn A, et al. Human cardiosphere-derived cells from advanced heart failure patients exhibit augmented functional is potency in myocardial repair. JACC Heart Fail. 2014; 2(1):49-61.
19. Mishra R, Vijayan K, Colletti E J, Harrington D A, Matthiesen T S, Simpson D, et al. Characterization and functionality of cardiac progenitor cells in congenital heart patients. Circulation. 2011; 123(4):364-73.
20. Makkar R R, Smith R R, Cheng K, Malliaras K, Thomson L E, Berman D, et al. Intracoronary cardiosphere-derived cells for heart regeneration after myocardial infarction (CADUCEUS): a prospective, randomised phase 1 trial. Lancet. 2012; 379(9819):895-904.
21. Chemyshev V S, Rachamadugu R, Tseng Y H, Belnap D M, Jia Y, Branch K J, et al. Size and shape characterization of hydrated and desiccated exosomes. Anal Bioanal Chem. 2015; 407(12):3285-301.
22. Kolk M V, Meyberg D, Deuse T, Tang-Quan K R, Robbins R C, Reichenspumer H, et al. LAD-ligation: a murine model of myocardial infarction. J Vis Exp. 2009(32).
23. Cash J L, White G E, Greaves D R. Chapter 17. Zymosan-induced peritonitis as a simple experimental system for the study of inflammation. Methods Enzymol. 2009; 461:379-96.
24. Dang T M, Wong W C, Ong S M, Li P, Lum J, Chen J, et al. MicroRNA expression profiling of human blood monocyte subsets highlights functional differences. Immunology. 2015; 145(3):404-16.
25. Driedonks TAP, Nolte-'t Hoen ENM. Circulating Y-RNAs in Extracellular Vesicles and Ribonucleoprotein Complexes; Implications for the Immune System. Front Immunol. 2018; 9:3164.
26. Kowalski M P, Krude T. Functional roles of non-coding Y RNAs. Int J Biochem Cell Biol. 2015; 66:20-9.
27. Hendrick J P, Wolin S L, Rinke J, Lemer M R, Steitz J A. Ro small cytoplasmic ribonucleoproteins are a subclass of La ribonucleoproteins: further characterization of the Ro and La small ribonucleoproteins from uninfected mammalian cells. Mol Cell Biol. 1981; 1(12):1138-49.
28. Lemer M R, Boyle J A, Hardin J A, Steitz J A. Two novel classes of small ribonucleoproteins detected by antibodies associated with lupus erythematosus. Science. 1981; 211(4480):400-2.
29. Nolte-'t Hoen E N, Buermans H P, Waasdorp M, Stoorvogel W, Wauben M H, t Hoen P A. Deep sequencing of RNA from immune cell-derived vesicles uncovers the is selective incorporation of small non-coding RNA biotypes with potential regulatory functions. Nucleic Acids Res. 2012; 40(18):9272-85.
30. Tosar J P, Gambaro F, Sanguinetti J, Bonilla B, Witwer K W, Cayota A. Assessment of small RNA sorting into different extracellular fractions revealed by high-throughput sequencing of breast cell lines. Nucleic Acids Res. 2015; 43(11):5601-16.
31. Wei Z, Batagov A O, Schinelli S, Wang J, Wang Y, El Fatimy R, et al. Coding and noncoding landscape of extracellular RNA released by human glioma stem cells. Nat Commun. 2017; 8(1):1145.
32. Haderk F, Schulz R, Iskar M, Cid L L, Worst T, Willmund K V, et al. Tumorderived exosomes modulate P D-L 1 expression in monocytes. Sci Immunol. 2017; 2(13).
33. Riley J L. PD-1 signaling in primary T cells. Immunol Rev. 2009; 229(1):114-25.
34. Chimenti I, Smith R R, Li T S, Gerstenblith G, Messina E, Giacomello A, et al. Relative roles of direct regeneration versus paracrine effects of human cardiosphere-derived cells transplanted into infarcted mice. Circ Res. 2010; 106(5):971-80.
35. Lee S T, White A J, Matsushita S, Malliaras K, Steenbergen C, Zhang Y, et al. Intramyocardial injection of autologous cardiospheres or cardiosphere-derived cells preserves function and minimizes adverse ventricular remodeling in pigs with heart failure post-myocardial infarction. J Am Coll Cardiol. 2011; 57(4):455-65.
36. Li T S, Cheng K, Lee S T, Matsushita S, Davis D, Malliaras K, et al. Cardiospheres recapitulate a niche-like microenvironment rich in sternness and cell-matrix interactions, rationalizing their enhanced functional potency for myocardial repair. Stem Cells. 2010; 28(11):2088-98.
37. Stastna M, Chimenti I, Marban E, Van Eyk J E. Identification and functionality of proteomes secreted by rat cardiac stem cells and neonatal cardiomyocytes. Proteomics. 2010; 10(2):245-53.
38. Gallet R, Dawkins J, Valle J, Simsolo E, de Couto G, Middleton R, et al. Exosomes secreted by cardiosphere-derived cells reduce scarring, attenuate adverse remodelling, and improve function in acute and chronic porcine myocardial infarction. Eur Heart J. 2017; 38(3):201-11.
39. Blazquez R, Sanchez-Margallo F M, Crisostomo V, Baez C, Maestre J, is Alvarez V, et al. Intrapericardial Delivery of Cardiosphere-Derived Cells: An Immunological Study in a Clinically Relevant Large Animal Model. PLoS One. 2016; 11(2):e0149001.
40. Middleton R C, Fournier M, Xu X, Marban E, Lewis M I. Therapeutic benefits of intravenous cardiosphere-derived cell therapy in rats with pulmonary hypertension. PLoS One. 2017; 12(8):e0183557.
41. Liu T, Zhang L, Joo D, Sun S C. N F-kappaB signaling in inflammation. Signal Transduct Target Ther. 2017; 2.
42. Langmead B, Trapnell C, Pop M, Salzberg S L. Ultrafast and memory-efficient alignment of short DNA sequences to the human genome. Genome Biol. 2009; 10(3):R25.
43. Friedlander M R, Chen W, Adamidi C, Maaskola J, Einspanier R, Knespel S, et al. Discovering microRNAs from deep sequencing data using miRDeep. Nat Biotechnol. 2008; 26(4):407-15.
44. Li H, Durbin R. Fast and accurate long-read alignment with Burrows-Wheeler transform. Bioinformatics. 2010; 26(5):589-95.

1-90. (canceled)

91. A plurality of engineered extracellular vesicles, wherein the extracellular vesicles comprise miR-345, and wherein the amount of miR-345 in the engineered extracellular vesicles is substantially higher than an amount of miR-345 in non-engineered extracellular vesicles.

92. The plurality of engineered extracellular vesicles of claim 91, wherein the extracellular vesicles further comprise miR-146a, and wherein the amounts of the miR-146a in the engineered extracellular vesicles are substantially higher than the amounts of miR-146a in non-engineered extracellular vesicles.

93. The plurality of engineered extracellular vesicles of claim 91, wherein the extracellular vesicles further comprise let-7b, and wherein the amounts of let-7b in the engineered extracellular vesicles are substantially higher than the amounts of let-7b in non-engineered extracellular vesicles.

94. The plurality of engineered extracellular vesicles of claim 92, wherein the extracellular vesicles further comprise let-7b, and wherein the amounts of let-7b in the engineered extracellular vesicles are substantially higher than the amounts of let-7b in non-engineered extracellular vesicles.

95. The plurality of engineered extracellular vesicles of claim 91, wherein the amounts of miR-345 in the engineered extracellular vesicles are at least 2-fold, 3-fold, 4-fold, 5-fold, 6-fold, 7-fold, 8-fold, 9-fold, 10-fold, 15-fold, or 20-fold higher than the amounts of miR-345 in non-engineered extracellular vesicles.

96. The plurality of engineered extracellular vesicles of claim 91, wherein the amount of miR-10b in the engineered extracellular vesicles is substantially lower than the amount of miR-10b in non-engineered extracellular vesicles derived from the cells.

97. The plurality of engineered extracellular vesicles according to claim 96, wherein the amount of miR-10b in the engineered extracellular vesicles is at least 2-fold, 3-fold, 4-fold, 5-fold, 6-fold, 7-fold, 8-fold, 9-fold, 10-fold, 15-fold, or 20-fold lower than the amount of miR-10b in non-engineered extracellular vesicles derived from the cells.

98. The plurality of engineered extracellular vesicles of claim 91, wherein the extracellular vesicles are exosomes or microvesicles.

99. The engineered extracellular vesicles of claim 98, wherein the extracellular vesicles are derived from cardiosphere-derived cells (CDCs), mesenchymal stromal/stem cells (MSCs), or 293F cells.

100. A method of producing engineered extracellular vesicles, the method comprising (a) genetically modifying a plurality of cells to overexpress miR-345, whereby the engineered extracellular vesicles produced by the cells comprise a substantially greater amount of miR-345 than extracellular vesicles produced by cells not so genetically modified, (b) or loading extracellular vesicles with miR-345, whereby the engineered extracellular vesicles comprise a substantially greater amount of miR-345 than extracellular vesicles not so loaded.

101. The method of producing engineered extracellular vesicles according to claim 100, the method further comprising (a) loading extracellular vesicles with miR-146a, whereby the engineered extracellular vesicles comprise substantially greater amounts of miR-146a than extracellular vesicles not so loaded, or (b) genetically modifying the plurality of cells to overexpress miR-146a, whereby the engineered extracellular vesicles produced by the cells comprise substantially greater amounts of miR-146a than extracellular vesicles produced by cells not so genetically modified.

102. The method of producing engineered extracellular vesicles according to claim 100, the method further comprising (a) loading extracellular vesicles with let-7b, whereby the engineered extracellular vesicles comprise substantially greater amounts of let-7b than extracellular vesicles not so loaded, or (b) genetically modifying a plurality of cells to overexpress let-7b, whereby the engineered extracellular vesicles produced by the cells comprise substantially greater amounts of let-7b than extracellular vesicles produced by cells not so genetically modified.

103. The method of producing engineered extracellular vesicles according to claim 101, the method further comprising (a) loading extracellular vesicles with let-7b, whereby the engineered extracellular vesicles comprise substantially greater amounts of let-7b than extracellular vesicles not so loaded, or (b) genetically modifying a plurality of cells to overexpress let-7b, whereby the engineered extracellular vesicles produced by the cells comprise substantially greater amounts of let-7b than extracellular vesicles produced by cells not so genetically modified.

104. The method according to claim 100, wherein said genetic modification of cells is effected via transfection or transduction of an expression vector encoding miR-345 into the cells, and/or via CRISPR/Cas9 gene editing of the cells.

105. The method according to claim 105, wherein said expression vector comprises an expression control sequence.

106. The method according to claim 100, wherein said loading of extracellular vesicles is effected with a chemical lipofection reagent or a chemical transfection reagent.

107. The method according to claim 100, wherein the amount of miR-345 in the engineered extracellular vesicles are at least 2-fold, 3-fold, 4-fold, 5-fold, 6-fold, 7-fold, 8-fold, 9-fold, 10-fold, 15-fold, or 20-fold higher than the amount of miR-345 in non-engineered extracellular vesicles derived from the cells.

108. The method according to claim 100, further comprising substantially depleting the extracellular vesicles of miR-10b, whereby the amount of miR-10b in the engineered extracellular vesicles is substantially lower than the amount of miR-10b in non-engineered extracellular vesicles.

109. The method according to claim 108, wherein the amount of miR-10b in the engineered extracellular vesicles is at least 2-fold, 3-fold, 4-fold, 5-fold, 6-fold, 7-fold, 8-fold, 9-fold, 10-fold, 15-fold, or 20-fold lower than the amount of miR-10b in non-engineered extracellular vesicles derived from the cells.

110. The method according to claim 100, wherein the extracellular vesicles are exosomes or microvesicles and the cells are cardiosphere-derived cells (CDCs), mesenchymal stromal/stem cells (MSCs), or 293F cells.
